(19) United States
(12) Patent Application Publication
(10) Pub. No.: US 2011/0236978 A1
(43) Pub. Date: Sep. 29, 2011

(54) REPROGRAMMING CELLS TOWARD A PLURIPOTENT STATE

(75) Inventors: Alexandra Stolzing, Leipzig (DE); Antje Arnold, Leipzig (DE); Jeremy Brown, Munchen (DE)

(73) Assignee: Fraunhofer-Gesellschaft zur Forderung der angewandten Forschung e. V., Munchen (DE)

(21) Appl. No.: 13/130,093

(22) PCT Filed: Nov. 17, 2009
(86) PCT No.: PCT/EP2009/008170

§ 371 Date: May 19, 2011

(30) Foreign Application Priority Data
Nov. 21, 2008 (EP) 08400048.8

(51) Int. Cl. C12N 15/11

(52) U.S. Cl. 435/455; 435/440

(57) ABSTRACT

The present invention relates to a process for preparing reprogrammed cells, in particular for preparing pluripotent and multipotent stem cells and to the pluripotent and multipotent stem cells prepared by said processes.

The present invention relates to a process for preparing reprogrammed cells, in particular for preparing pluripotent and multipotent stem cells and to the pluripotent and multipotent stem cells prepared by said processes.

Aging—whether in vivo during the life course or in vitro during scale up—affects cells, in particular stem cells. For example, aged cells show increased apoptosis rates, decreased differentiation and proliferation capacity, and decreased therapeutic effectiveness. During aging, stem cells often differentiate into a less pliable, less proliferative form. Cell aging represents a problem in particular when using cells from elderly patients/donors as starting material for cell cultures, when expanding cells in vitro and when using cells that have a limited differentiation capacity for therapy or research.

One approach to dedifferentiate aged cells is to create so called Induced Pluripotent Stem Cells (IPS). The current state of the art foresees to use a viral transfer of transcription factors to induce increased differentiation potency in cells and to rejuvenate them. This, however, is potentially dangerous because a viral transfer permanently changes the genome of the cell and also might lead to complications because of the viral parts left behind in the cell genome. Moreover, viral integration into the genome is known to increase cancer risk in cells. Essentially the same applies to the use of plasmids for reprogramming cells (Okita et al., 2008, Science. 2008 Nov. 7; 322(5903):949-53. Generation of mouse induced pluripotent stem cells without viral vectors).

Another approach employs contacting cells to be reprogrammed with extracts of human embryonic stem cells for reprogramming cells. This approach, however, is ethically and practically problematic.

Some other approaches use chemical factors to dedifferentiate cells (e. g. US 2007/0254884 A1, US 2007/0020759 A1), but these factors are not efficacious in fully reprogramming cells to a pluripotent state by themselves.

Thus, the technical problem underlying the present invention is to provide improved methods to reprogram cells, in particular to dedifferentiate cells, which overcome the above-identified disadvantages, in particular allow in an easy, efficient and safe way the provision of reprogrammed, in particular dedifferentiated cells, preferably displaying multipotent, most preferably pluripotent, stem cell character.

The present invention solves its technical problem by the provision of a process for preparing reprogrammed cells in vitro, comprising the process steps, preferably in the given order, a) providing cells to be reprogrammed; b) introducing, preferably transfecting, mRNA molecules capable of reprogramming the cells into the cells provided in step a), wherein the reprogramming mRNA molecules are coding for at least one protein selected from the group consisting of: Ronin, Oct4, Klf4, Sox2, Nanog and TERT; and c) culturing the cells obtained in step b) in a cell culture medium and under a condition suitable to allow translation of the introduced, preferably transfected reprogramming mRNA molecules so as to obtain reprogrammed cells. In a preferred embodiment the reprogramming mRNA molecules of the present invention are coding for at least one protein selected from the group consisting of Ronin, Oct4, Kef4, Sox2 and TERT.

Thus, the present invention foresees a process which is able to prepare reprogrammed cells without the need to permanently change the genome of the cell and without the need to use viruses to transfer transcription factors or the like into the cell. The present invention also is advantageous in so far, as for reprogramming cells no extracts of human embryonic stem cells or cost intensive and potentially dangerous chemical factors are needed. Thus, ethical concerns, genetic stability problems and cancer risks are reduced or even avoided. In contrast, the present invention provides the advantageous teaching to introduce, preferably transfect, specific mRNA molecules, in the following termed “reprogramming mRNA molecules”, into the cells, wherein these reprogramming mRNA molecules are able to reprogram the mRNA recipient cells, preferably the cells being transfected therewith, into a dedifferentiated status. Advantageously, the mRNA molecules introduced, preferably transfected, according to the present invention will not integrate into the recipient's genome and therefore do not pose a risk of cancer or genetic instability. The invention therefore foresees a process for preparing one or more reprogrammed cells in vitro wherein said process does not include any cloning amplification or proliferation of the mRNA recipient cells. The present invention also foresees a process for preparing one or more reprogrammed cells in vivo, that is in a living organ, a living organism or animal, more particular a mammal.

In the context of the present invention the term “reprogramming” preferably means remodelling, in particular erasing and/or remodelling, epigenetic marks of a cell such as DNA methylation, histon methylation or activating genes by inducing transcription factor signal systems as for oct4. In particular, the reprogramming of the present invention provides at least one dedifferentiated and/or rejuvenated cell, in particular provides a cell having the characteristic of a multipotent, in particular pluripotent stem cell. Thus, in case the cell to be reprogrammed is cells which already have a multipotent or pluripotent character, the present invention is able to maintain these cells by the reprogramming of the present invention in their multi- or pluripotent state for a prolonged period of time. In case the cells to be reprogrammed are in an aged or differentiated state, the present invention allows the dedifferentiation into a multipotent or pluripotent stem cell. In a particularly preferred embodiment, multipotent cells may be reprogrammed to become pluripotent cells.

In particular, the term “reprogrammed cells” designates cells which have been changed to have a higher differentiation and/or proliferation potential. Also, in a preferred embodiment the reprogramming of a somatic differentiated cell toward a multipotent stem cell or “young” differentiated cell is termed reprogramming and the product is called a reprogrammed cell.

In particular, the present process allows it to enrich immature stem cells, preferably showing a high telomerase activity, several multipotent, preferably pluripotency, markers and an increased secretion of growth factors. The present invention provides the advantage that aged stem cells can be expanded for a longer period of time and with a higher stem cell yield. Further, stem cell lines and tissue engineered construct drafts made or derived from cells reprogrammed by the present process show a reduced minimal immunological risk, in particular if the patient owns cells are used as a source for the reprogramming. Thus, the present invention provides means and methods to prepare in particular pluripotent stem cells, to provide multipotent stem cells, to provide means and methods for an increased expansion of cells or stem cells in vitro and for the rejuvenation of aged cells or aged stem cells for tissue engineering or cell therapies.

In particular, the present invention foresees, preferably in a first step, to provide cells to be reprogrammed. Preferably, the cells to be reprogrammed may be adult, neonatal or embryonic differentiated cells, but are not limited thereto. Preferably, the cells to be reprogrammed are adult or neonatal undifferentiated cells. All of these cells may, in a preferred embodiment, be cell lines, immortalized cells, cells kept in cell culture, isolated cell populations, preferably cells isolated from a donor, either a living or dead donor, or cells isolated from the environment. Cell types which can be reprogrammed according to the present invention potentially are all cell types, and are in preferred instances fibroblasts, hepatocytes, cardiac cells, cardiomyocytes, nerve cells, chondrocytes, osteoblasts, adipocytes, myoblasts, hepatoblasts, hepatic stem cells, insulin producing cells, neural stem cells, cardiomyogenic cells, dermatocytes, keratinocytes, pancreatic cells, monocytes, epithelial cells or mesenchymal stem cells (MSC). Preferably, the cells may be mammalian cells, in particular human cells or animal cells, preferably ovine, horse, ape or in particular rodent cells, preferably hamster cells, mouse cells or rat cells. The cells may also be fish, reptile, avian, amphibian or insect cells. Preferably, the cells to be reprogrammed are lineage-committed cells. In a further preferred embodiment the cells, in particular the cells of the above identified origin, are stem cells, in particular post-natal stem cells or non-embryonic stem cells.

A suitable and preferred stem cell source like mesenchymal stem cells is a tissue within the human or animal body which comprises the stem cells for use in the present invention, optionally together with other cell types. Preferably, the suitable stem cell source is bone marrow, both adult and fetal, cytokine or chemotherapy mobilized peripheral blood, fetal liver, umbilical cord blood, embryonic yolk sac and spleen, both adult and fetal, more preferably the stem cell source is adult bone marrow or umbilical cord blood, most preferably the stem cell source is bone marrow. Bone marrow cells may be obtained from any known source, including, but not limited to, ilium, sternu, tibiae, femora, spine or other bone cavities. Preferably, the stem cells are isolated from a mammalian organism such as human, mouse or rat, and more preferably the stem cells are isolated from a human organism.

The term “isolated cell population” is intended to mean that the cells are not in contact with other cells with which they are usually in contact within the body of the mammal or in a tissue sample obtained directly, i.e. without purification or enrichment step, from the mammal. A “cell population” according to the present invention may comprise not only cells of one cell type such as fibroblasts or mesenchymal stem cells as defined for example by the expression of a specific combination of surface markers, but also a mixture of cells of different cell types which show different combinations of surface markers.

The cells harvested from said sources may be used directly for transfection or may be cryoconserved by freezing them at a temperature from about −196° C. to about −130° C.

The cells to be reprogrammed receive, according to the present invention, in a second step at least one species of reprogramming mRNA molecules, in particular linear and isolated mRNA molecules, selected from the group of mRNA molecules encoding Ronin, mRNA molecules encoding Oct4, mRNA molecules encoding Klf4, mRNA molecules encoding Sox2, mRNA molecules encoding Nanog and mRNA molecules encoding TERT. Ronin, Oct4, Klf4, Sox2, Nanog and TERT are proteins with the ability to reprogram cells. In the context of the present invention these proteins are termed “reprogramming proteins” and are characterised by their ability to reprogram target cells and therefore by their regulatory function in terms of determining the cell fate, in particular being able to dedifferentiate a target cell and/or maintain a cell in a dedifferentiated state, preferably by being transcription factors.

Differentiation behaviour of cells and state of differentiation can be detected according to the methods set forth below. However any method for determining the differentiation state or potency of a cell known the skilled person are applicable as well.

In a preferred embodiment, the cell to be reprogrammed is transfected with one or more reprogramming mRNA molecules according to the present invention. In another preferred embodiment the cell to be reprogrammed is supplemented with one or more reprogramming mRNA molecules according to the present invention. In another preferred embodiment the cell to be reprogrammed is injected with one or more reprogramming mRNA molecules according to the present invention.

The present invention thus foresees to use one or more of mRNA molecules encoding Ronin, Oct4, Klf4, Sox2, Nanog or TERT. These mRNA molecules may, in a preferred embodiment, be molecules having the wild-type mammalian, in particular human, animal, preferably rodent, more preferably mouse, hamster or rat or any other animal nucleotide sequence. In a preferred embodiment of the present invention the reprogramming mRNA molecule is selected from the group consisting of mRNA nucleotide sequence molecules being encoded by any one of the DNA sequences given under SEQ ID No. 1 to 16.

Of course, the invention also foresees to use mRNA molecules, whose sequence is a functional equivalent to the polynucleotide sequence given above. In the context of the present invention a functional equivalent is a nucleotide sequence molecule, which either codes with a different nucleotide sequence exactly the same protein as the mRNA sequence encoded by any one of SEQ ID No. 1 to 16 or is a mRNA sequence, which encodes a protein with a different amino acid sequence but with same or similar function, in particular being able to reprogram the cells according to the present invention.

In a particularly preferred embodiment of the present invention the functional equivalent of the mRNA molecules is a polynucleotide with a homologous sequence to the wild-type mRNA polynucleotide as encoded by the DNA sequence in any one of SEQ ID No. 1 to 16. In a preferred embodiment of the present invention the degree or percentage of homology is at least 50, 60, 70, 80, 90, 95, 99 or 100%. In a furthermore preferred embodiment a functional equivalent of the mRNA molecules as given in any one of SEQ ID No. 1 to 16 is a mRNA sequence which is encoded by a DNA sequence being substantially complementary to the sequences of any one of SEQ ID No. 1 to 16 or to its complementary, preferably substantially, complementary sequence. In a furthermore preferred embodiment the functional equivalent of the mRNA molecule as encoded by any one of the DNA sequences of SEQ ID No. 1 to 16 is a mRNA molecules being encoded by a DNA sequence which is able to hybridize under stringent or reduced stringent conditions to any one of the polynucleotides given in SEQ ID No. 1 to 16 or to its complementary, preferably substantially, complementary sequence.

In the context of the present invention the term “mRNA molecule” is meant to refer to a linear polymer of ribonucleotide molecules, which is single-stranged and serves as a template for protein synthesis.

Polynucleotides have “homologous” sequences if the sequence of nucleotides in the two sequences is the same when aligned for maximum correspondence as described herein. Sequence comparison between two or more polynucleotides is generally performed by comparing portions of the two sequences over a comparison window to identify and compare local regions of sequence similarity. The comparison window is generally from about 20 to 200 contiguous nucleotides.

The “percentage of sequence homology” for polynucleotides, herein referred to be 50, 60, 70, 80, 90, 95, 98, 99 or 100 percent sequence homology, may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology.

Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul, S. F. et al. 1990. J. Mol. Biol. 215:403; Altschul, S. F. et al. 1997. Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

As used herein, “substantially complementary” means that two nucleic acid sequences in question have at least about 65%, preferably about 70%, more preferably about 80%, even more preferably 90%, and most preferably about 98%, sequence complementarity to each other. This means that the DNA sequence coding the functional equivalent and the polynucleotide given in any of SEQ ID No. 1 to 16 or its complement must in a preferred embodiment exhibit sufficient complementarity to hybridise under stringent conditions. A substantially complementary sequence is preferably one that has sufficient sequence complementarity to the nucleotide sequence in question to result in binding.

The term “primer” as used herein refers to an oligonucleotide which is capable of annealing to the amplification target allowing a DNA polymerase to attach thereby serving as a point of initiation of DNA synthesis when placed under conditions in which synthesis of primer extension product which is complementary to a nucleic acid strand is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxy ribonucleotide. The primer must be sufficiently long to prime the synthesis of extension products in the presence of the agent for polymerization. The exact lengths of the primers will depend on many factors, including temperature and source of primer.

A “pair of bi-directional primers” as used herein refers to one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimised to maximize specific binding and minimize nonspecific binding of an oligo- or polynucleotide to its target nucleic acid sequence. The terms as used include reference to conditions under which a an oligo- or polynucleotide will hybridise to its target sequence, to a detectably greater degree than other sequences (e.g. at least 2-fold over background). Stringent conditions are sequence dependent and will be different in different circumstances. Longer sequences hybridise specifically at higher temperatures.

Generally, stringent conditions are selected to be about 5 DEG C. (degree celcius) lower than the thermal melting point (Tm) for the specific sequence at a defined ionic strength and pH. The Tm is the temperature (under defined ionic strength and pH) at which 50% of a complementary target sequence hybridises to a perfectly matched oligo- or polynucleotide. Typically, stringent conditions will be those in which the salt concentration is less than about 1.0 M Na <+> ion, typically about 0.01 to 1.0 M Na <+> ion concentration (or other salts) at pH 7.0 to 8.3 and the temperature is at least about 30 DEG C. for short probes or primers (e.g. 10 to 50 nucleotides) and at least about 60 DEG C. for long probes or primers (e.g. greater than 50 nucleotides). Stringent conditions may also be achieved with the addition of destabilizing agents such as formamide.

Exemplary low stringent conditions or “conditions of reduced stringency” include hybridization with a buffer solution of 30% formamide, 1 M NaCl, 1% SDS at 37 DEG C. and a wash in 2×SSC at 40 DEG C. Exemplary high stringency conditions include hybridization in 50% formamide, 1 M NaCl, 1% SDS at 37 DEG C., and a wash in 0.1×SSC at 60 DEG C. Hybridization procedures are well known in the art and are described in e.g. Ausubel et al, Current Protocols in Molecular Biology, John Wiley & Sons Inc., 1994.

Thus, the present invention foresees to introduce, preferably transfect, specific reprogramming mRNA molecules into the cells, which mRNA molecules once introduced, preferably transfected, in the cells are able to be translated into so called reprogramming proteins which can turn on specific genes in the cells, in particular multipotency and/or the pluripotency genes in the cell. The mRNA molecule used according to the present invention is preferably a linear molecule having a poly A tail, most preferably produced by in vitro transcription. Thus, in a preferred embodiment the mRNA molecules are produced by in vitro transcription, in particular using bacterial systems. In a particular preferred embodiment the DNA sequences of the reprogramming proteins are cloned into plasmids and amplified in bacteria, for instance E. coli. In a further preferred embodiment, the plasmids are then isolated from the bacteria, are linearised and subjected to restriction digestion. In a further preferred embodiment cDNA prepared by said method is transcribed into mRNA, which in turn is incubated to destroy cDNA residues and to obtain mRNA molecules to be introduced into the cells.

Since the introduced reprogramming mRNA molecules and the proteins translated therefrom are over the time being degraded in the cells, they cannot permanently integrate into the genome of the cell providing some of the advantageous features of the present invention.

In a preferred embodiment of the present invention the present teaching foresees to introduce, preferably transfect, the reprogramming mRNA molecules into the cell by electroporation, by lipofection, by injection, by magnetofection, by particle bombardment, gene gun, or by any other method known in the art suitable to introduce mRNA molecules into a target cell.

In a particular preferred embodiment the present invention further foresees in its step c) to culture the cells into which the reprogramming mRNA molecules have been introduced in a cell culture medium and under a condition suitable to allow translation of the transfecting reprogramming mRNA molecules, so as to obtain the reprogrammed cells.

In a particularly preferred embodiment of the present invention the present process therefore consists of the above-identified steps a), b) and c), preferably in this order. Accordingly, the present invention excludes any further process steps, in particular any further substantial process step, in particular any intervening or subsequent process steps. Thus, the present invention provides its advantages in a simple and cost-effective way.

In a particularly preferred embodiment the cell culture system is a cell culture system, comprising a cell culture medium, preferably in a culture vessel, in particular a cell culture medium supplemented with at least one so called “inducing substance”, which is a substance suitable and determined for protecting the cells from in vitro aging and/or inducing in an unspecific or specific reprogramming. In a particularly preferred embodiment an inducing substance according to the present invention is a substance selected from the group consisting of reversin, resveratrol, selenium, a selenium-containing compound, EGCG ((−)-epigallocatechin-3-gallate), valproic acid and salts of valproic adic, in particular sodium valproate.

In a particularly preferred embodiment the at least one inducing substance is present in the cell culture medium used in step c) of the present process in a concentration from 0.001 to 100 μM, preferably from 0.005 to 50 μM.

In a particularly preferred embodiment the present invention foresees to use a concentration of reversin from 0.5 to 10 μM, preferably of 1 μM. In a furthermore preferred embodiment the present invention foresees to use resveratrol in a concentration of 10 to 100 μM, preferably 50 μM. In a furthermore preferred embodiment the present invention foresees to use selenium or a selenium containing compound in a concentration from 0.05 to 0.5 μM, preferably of 0.1 μM. In furthermore preferred embodiment the present invention foresees to use EGCG in a concentration from 0.001 to 0.1 μM, preferably of 0.01 μM. In a furthermore preferred embodiment the present invention foresees to use valproic acid or sodium valproate in a concentration from 1 to 10 μM, in particular of 5 μM.

The present invention foresees in a furthermore preferred embodiment culturing the cells obtained in step b) in a cell culture medium, wherein the cell culture medium comprises, optionally in combination with the inducing substance as specified above, at least one transient proteolysis inhibitor. The use of at least one proteolysis inhibitor in the cell culture medium of the present invention increases the time the reprogramming proteins derived from the mRNA or any endogenous genes will be present in the cells and thus facilitates in an even more improved way the reprogramming by the transfected mRNA derived factors. The present invention uses in a particularly preferred embodiment as a transient proteolysis inhibitor a protease inhibitor, a proteasome inhibitor and/or a lysosome inhibitor. In a particularly preferred embodiment the proteosome inhibitor is selected from the group consisting of MG132, TMC-95A, TS-341 and MG262.

In a furthermore preferred embodiment the protease inhibitor is selected from the group consisting of aprotinin, G-64 and leupeptine-hemisulfat. In a furthermore preferred embodiment the lysosomal inhibitor is ammonium chloride.

In a furthermore preferred embodiment the present invention also foresees a cell culture medium comprising at least one transient inhibitor of mRNA degradation. The use of a transient inhibitor of mRNA degradation increases the half-life of the reprogramming factors as well.

In a furthermore preferred embodiment of the present invention a condition suitable to allow translation of the transfected reprogramming mRNA molecules in the cells is an oxygen content in the cell culture medium from 0.5 to 21%, preferably from 1 to 20%, more preferably from 5 to 19% and particularly preferred from 10 to 18%. More particular, and without wishing to be bound to the theory, oxygen is used to further induce or increase Oct4 by triggering Oct4 via Hif1a.

In a preferred embodiment conditions that are suitable to support reprogramming of the cells by the mRNA molecules in the cells are selected; more particularly, these conditions require a temperature from 30 to 38° C., preferably from 31 to 37° C., most preferably from 32 to 36° C.

The glucose content of the medium is in a preferred embodiment of the present invention below 4.6 g/l, preferably below 4.5 g/l, more preferably below 4 g/l, even more preferably below 3 g/l, particularly preferably below 2 g/I and most preferably it is 1 g/l. DMEM media containing 1 g/l glucose being preferred for the present invention are commercially available as “DMEM low glucose” from companies such as PAA, Omega Scientific, Perbio and Biosera. More particular, and without wishing to be bound to the theory, high glucose conditions adversely support aging of cells (methylation, epigenetics) in vitro which may render the reprogramming difficult.

In a furthermore preferred embodiment of the present invention the cell culture medium contains glucose in a concentration from 0.1 g/l to 4.6 g/l, preferably from 0.5 g/l to 4.5 g/l and most preferably from 1 g/l to 4 g/l.

The terms “cell culture” and “culturing of cells” refer to the maintenance and propagation of cells and preferably human, human-derived and animal cells in vitro.

“Cell culture medium” is used for the maintenance of cells in culture in vitro. For some cell types, the medium may also be sufficient to support the proliferation of the cells in culture. A medium according to the present invention provides nutrients such as energy sources, amino acids and anorganic ions. Additionally, it may contain a dye like phenol red, sodium pyruvate, several vitamins, free fatty acids, antibiotics, anti-oxidants and trace elements.

For culturing the stem cells according to the present invention any standard medium such as Iscove's Modified Dulbecco's Media (IMDM), alpha-MEM, Dulbecco's Modified Eagle Media (DMEM), RPMI Media and McCoy's Medium is suitable before reprogramming. Ones the cells have been reprogrammed, they can in a preferred embodiment be cultured in embryonic stem cell medium.

Preferably, the medium additionally comprises one or more additives selected from the group consisting of vitamin D3 (1,25-dihydroxyvitamin D3, cacitriol), resveratrol (trans-3,4′,5-trihydroxystilbene), reversine (2-(4-morpholinoanilino)-N6-cyclohexyladenine), vitamin E (RRR-α-tocopherol), valproic acid (dekapene, valproate, valrelease), EGCG (epigallocatechin-3-gallat) and selenium. Preferably, two of the afore-mentioned additives are present, more preferably three of the afore-mentioned additives are present, even more preferably four of the afore-mentioned additives are present, particularly preferably five of the afore-mentioned additives are present and most preferably all of the afore-mentioned additives are present.

These media are well-known to a person skilled in the art and may be purchased from companies such as Cambrex, Invitrogen, Sigma-Aldrich or Stem Cell Technologies.

The medium may in a particular embodiment further comprise a serum component such as horse serum, human serum or fetal calf serum (FCS). Preferably, the medium contains FCS. If present, the serum is present in a concentration of 1-20%, preferably of 3-18%, more preferably of 5-15%, even more preferably of 8-12% and most preferably of 10%. Alternatively the serum component may be replaced by any of several standard serum replacement mixtures which typically include insulin, albumin and lecithin or cholesterol.

A “culture vessel” is any vessel which is suitable for growing cells in a culture medium, in particular selected from, but not limited to, agar, matrigel and collagen, either in fluid phase or adhered to an interior surface of the container. Types of such specialized vessels include roller bottles, spinner flasks, petri dishes and tissue flasks. Culture vessels are designed to be incubated in temperature, humidity and gas controlled environments to facilitate maximum cell or tissue growth. Generally, a layer of cell culture medium or agar covers the growing surface. The portion of the vessel not utilized as a growing surface encloses the interior gaseous environment which surrounds the cell culture. Cells, tissues, microorganisms and the like typically are introduced into the interior of cell culture vessels through an opening in the vessel. After introduction of the cells the opening may be closed such that the cells are not in contact with the environment during the culturing of the cells.

In a preferred embodiment of the present invention, the culture vessel is treated for tissue culture, which means that the surface of the culture vessel is treated such that cells which usually grow in an adherent state grow adherently, but cells which grow in suspension do not adhere or adhere only loosely. Such treatment may involve the irradiation of the vessel or the coating of the vessel, for example with a special plastic, polymer or nanostructure or with proteins of the extracellular matrix. Such vessels can comprise tissue culture dishes and tissue culture flasks and are available from different suppliers such as Becton Dickinson, Greiner, Sigma and TPP.

The present invention also relates to a process for the production of cells exhibiting a multipotent, preferably a multipotent, stem cell character, wherein a process according to the above is carried out.

The present invention also relates to a process for improving the expansion of stem cells in vitro, wherein a process according to the present invention is carried out. Thus, the present invention provides the advantage of prolonging expansion of stem cells, since the present invention keeps the cells to be expanded in a dedifferentiated status.

The present invention also relates to a process for the rejuvenation of aged cells, wherein a process according to the present invention is carried out.

The present invention also relates to a process for inducing the dedifferentiation of cells, wherein a process according to the present invention is carried out and wherein in particular the cells to be reprogrammed are differentiated cells, in particular lineage-committed cells.

The present invention also relates to a process for reprogramming recipient cells of a mammal in vivo comprising introducing reprogramming mRNA molecules into the recipient cells of the mammal wherein the reprogramming mRNA molecules are encoding at least one reprogramming protein selected from the group consisting of Ronin, Oct4, Klf4, Sox2, Nanog and TERT. Thus, the present invention also relates to an in vivo method for reprogramming recipient cells of a mammal which employ the reprogramming mRNA molecules as identified above for the in vitro embodiment of the present invention. The present invention relating to the process for reprogramming recipient cells of a mammal in vivo thereby foresees to introduce the reprogramming mRNA molecules directly into at least one cell of a recipient mammal that means into at least one recipient cell by suitable means such as gene gun insertion. Such a process allows the production of a mammal in particular a human being which has at least one reprogrammed cell. Thus, this embodiment of the present invention provides terrifically improved therapeutic application, in particular in the regenerative medicine and/or in replacement therapy. Thus, the present invention relates to a method of treating a mammal, in particular human being wherein reprogramming mRNA molecules are introduced into at least one cell of the recipient mammal and wherein the reprogrammed mRNA molecules are encoded at least by encoding at least one reprogramming protein selected from the group consisting of Ronin, Oct4, Klf4, Sox2, Nanog and TERT.

The present invention also relates to a multipotent, in particular a pluripotent, stem cell prepared according to any one of the methods of the present invention. Thus, the reprogrammed cells according to the present invention are preferably multipotent, in particular pluripotent stem cells. In a preferred embodiment the cells obtained according to the present invention are distinguished from the cells used as a starting material in step a) of the present invention by lack of one or more markers. These cells may in a preferred embodiment be also characterized by a particular methylation pattern, by the expression of oct4, sox2, nanog, hTERT or klf4. expression of oct4, which is not found in somatic cells like fibroblasts, becomes expressed after transfecting mRNA (klf4, sox2 and oct4) in human fibroblasts, by improved growth potential; and by increased differentiation potential. For pluripotency the formation of embryoid bodies, production of cells of all three germ layers in vitro and in vivo. Examples thereof are illustrated in the accompanying figures and example.

The reprogrammed cells obtained by the present processs may preferably be characterized by the expression or non-expression of certain surface markers. These surface markers are usually given a cluster of differentiation (CD) designation which describes groups of immunophenotypical surface characteristics of cells. Usually CD molecules are membrane-bound glycoproteins which are recognized by a cluster of monoclonal antibodies that display the same cellular reactivity. These surface molecules may for example be detected by means of a flow cytometer such as a fluorescence-activated cell sorter (FACS) manufactured by companies such as Becton Dickinson. Methods to identify cells according to the present invention are methods to detect cell-specific proteins, methods to detect cell-specific transcription factors or methods to detect physiological or morphological changes. All of these methods are per se well-known in the prior art. In particular, these methods use well-known protein or DNA or RNA detection processes and/or the reporter gene assays. Of course, immunoassays or assays using fluorescently or radioactively labelled proteins or nucleic acids including methods to measure enzymatic activities can be employed. Methods to detect morphological changes may include staining methods, visually based methods or the like.

Reprogrammed cells according to the present invention may be used for therapeutic, diagnostic or scientific purposes. In particular, these cells may be used in regenerative medicine and/or for replacement therapy.

The term “cell” not only refers to a single cell but also encompasses a cell line, a cell population or a cell clone.

The term “stem cells” refers to cells which have retained the capacity to proliferate and differentiate into one or different cell types. Stem cells created in accordance with the present invention are preferably pluripotent stem cells, i.e. stem cells which have retained the capacity to differentiate into distinct cell lineages and cell types or multipotent stem cells, retaining a more restricted differentiation potential.

It is understood that the term “stem cells” in accordance with the invention does not comprise human embryos. Furthermore, it is understood that the term “stem cells” does not comprise pluripotent stem cells which have been directly derived from a human embryo. Embryonic stem cells which have been derived from publicly available and previously established stem cell lines are understood to fall within the meaning of the term “stem cells” as used by the present invention.

A “stem cell”, as used herein, refers to any self-renewing pluripotent cell or multipotent cell or progenitor cell or precursor cell that is capable of differentiating into one or multiple cell types. Stem cells are thus cells able to differentiate into one or more than one cell type and have preferably an unlimited growth potential. Stem cells include those that are capable of differentiating into cells of osteoblast lineage, a mesenchymal cell lineage (e. g. bone, cartilage, adipose, muscle, stroma, including hematopoietic supportive stroma, and tendon). “Differentiate” or “differentiation”, as used herein, refers to the process by which precursor or progenitor cells (i. e., stem cells) differentiate into specific cell types, e. g., osteoblasts. Differentiated cells can be identified by their patterns of gene expression and cell surface protein expression. “Dedifferentiate” or “dedifferentiation”, as used herein, refers to the process by which lineage-committed cells (e. g., myoblasts or osteoblasts) reverse their lineage commitment and become precursor or progenitor cells (i. e., multipotent or pluripotent stem cells). Dedifferentiated cells can for instance be identified by loss of patterns of gene expression and cell surface protein expression associated with the lineage committed cells.

A “lineage-committed cell” as used herein, refers to any cell that has or will differentiate into a particular cell type or related cell types. Lineage committed cells include, for example, osteoblasts, myoblasts, chrondrocytes, and adipocytes.

Totipotent stem cells are able to create all cell types of the body, including placental cells. The earlier stage foetal stem cells are considered totipotent, as well as the fertilised egg. They also have the ability to replicate in unlimited numbers without losing their potential.

Pluripotent stem cells are able to create cells of all three germ layer, namely the ecto-, endo- and mesoderm. They also have the ability to replicate in unlimited numbers without losing their potential.

Multipotent stem cells are able to produce cells of one or more germ layers or several types of tissues. They often have an already limited self-renewal ability.

Stem cells are thus cells able to differentiate into one or more than one cell type and have preferably an unlimited growth potential.

Progenitor cells can differentiate into one or more cell types but have a limited growth potential.

Adult stem cells are stem cells derived from an adult organism and might be multipotent or pluripotent.

Embryonic stem cells are derived from the inner mass of a blastula and are pluripotent. Embryonic stem cells are unique because they can develop into nearly all cell types, an attribute called pluripotency. But to access these cells, researchers must destroy a viable embryo.

In this patent we are describing a way to create cells, with the characteristics of embryonic stem cells, without the need to destroy embryos or the use of embryonic stem cells.

Induced pluripotent stem cells “IPS” cells are pluripotent stem cells artificially derived from non-pluripotent cells, including somatic cells, adult multipotent stem cells or progenitor cells. In principal every cell containing a nuclei can be used as source for IPS cells.

Further preferred embodiments of the present invention are the subject matter of the subclaims.

The sequence listing show the prior art DNA sequences encoding the mRNA nucleotide sequence molecules used in the present invention:

TABLE 1

| SEQ ID No. | gene | accession-number | length |
|---|---|---|---|
| 1 | hNanog | NM_024865 | 2098 |
| 2 | mNanog | NM_028016 | 1356 |
| 3 | rNanog | NM_001100781 | 2358 |
| 4 | hSox2 | NM_003106 | 2518 |
| 5 | mSox2 | NM_011443 | 2457 |
| 6 | rSox2 | NM_001109181 | 2323 |
| 7 | hOct4 | NM_002701 | 1411 |
| 8 | mOct4 | NM_013633 | 1346 |
| 9 | hKlf4 | NM_004235 | 2949 |
| 10 | mKlf4 | NM_010637 | 3057 |
| 11 | rKlf4 | NM_053713 | 2393 |
| 12 | hTERT | NM_198253 | 4018 |
| 13 | mTert | ENSMUSG00000021611 | 4237 |
| 14 | rTERT | NM_053423 | 3378 |
| 15 | hRonin | NM_020457 | 1903 |
| 16 | mRonin | NM_021513 | 1832 |

abbreviations:
h = human;
m = mouse;
r = rat

The designations marked “NM” and ENSMUSG refer to the NM(NCBI)- and ENSMUSG-accession numbers as given under the publically available website http://www.ncib.nlm.nih.gov and http://www.ensembl.org.

SEQ ID No. 17 and 18 show the DNA sequence of primers used for cloning the human Nanog gene.

SEQ ID No. 19 and 20 show the DNA sequence of primers used for cloning the human Klf4 gene.

SEQ ID No. 21 and 22 show the DNA sequence of primers used for cloning the human Sox2 gene.

SEQ ID No. 23 and 24 show the DNA sequence of primers used for cloning the human Oct4 gene.

SEQ ID No. 25 and 26 show the DNA sequence of primers used for cloning the human Tert gene.

SEQ ID No. 27 and 28 show the DNA sequence of primers used for cloning the GFP gene.

The present invention will now be illustrated in more detail by way of an example and figures.

The figures show:

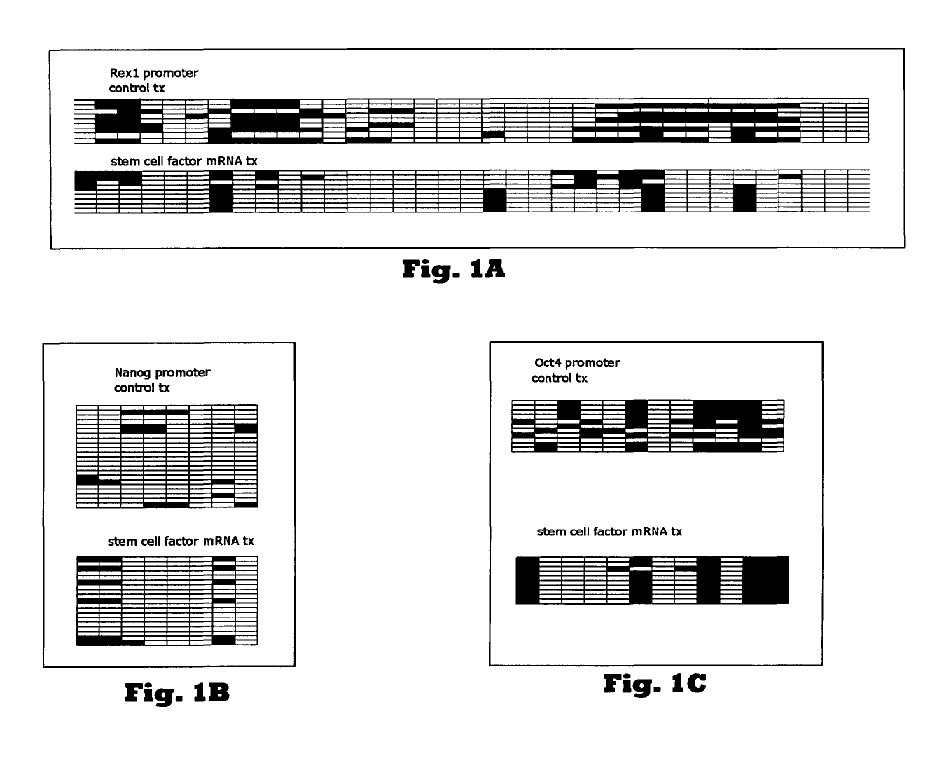

FIG. 1: Promotor methylation of rex1, nanog and oct4 in human fibroblasts before and after reprogramming (FIG. 1A: Rex; FIG. 1AB: Nanog; FIG. 1C: Oct4). CpG island are represented as squares. Black squares represent methylated and white squares represent un-methylated CpG islands. All samples are primary fibroblasts from a young male donor.

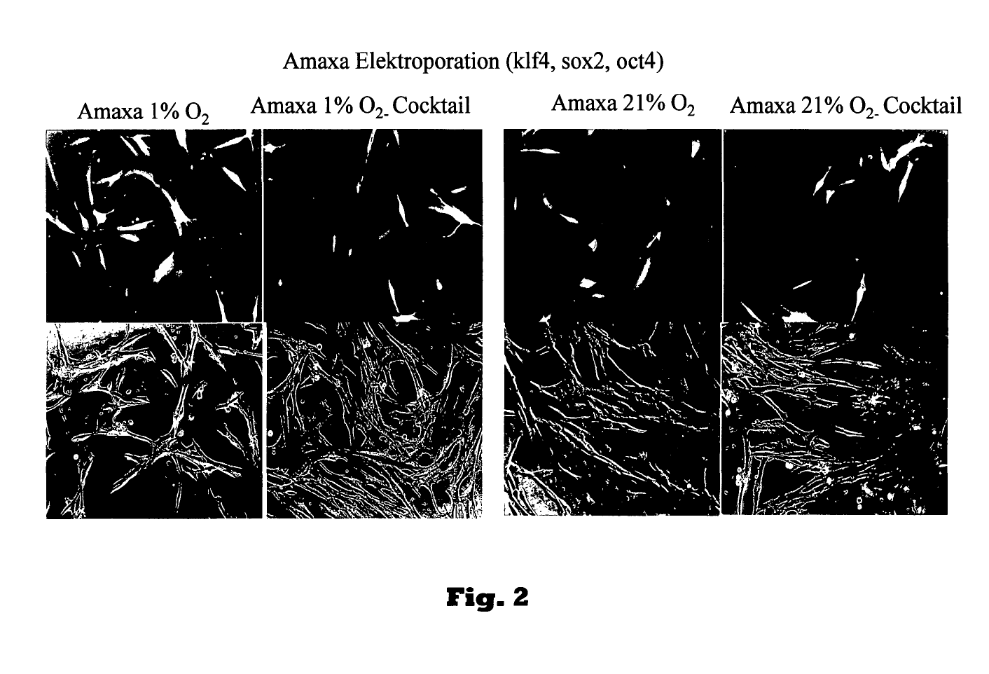

FIG. 2: Fluorescence and morphology after amaxa elektroporation (klf4, sox2, oct4): Human primary fibroblasts were transfected with 0.6 μg/μl mRNA per factor and 0.2 μg/μl GFP mRNA to visualize the transfection effciency. Top line are fluorescence pictures and the bottom line shows the morphology of the cells after 2 days.

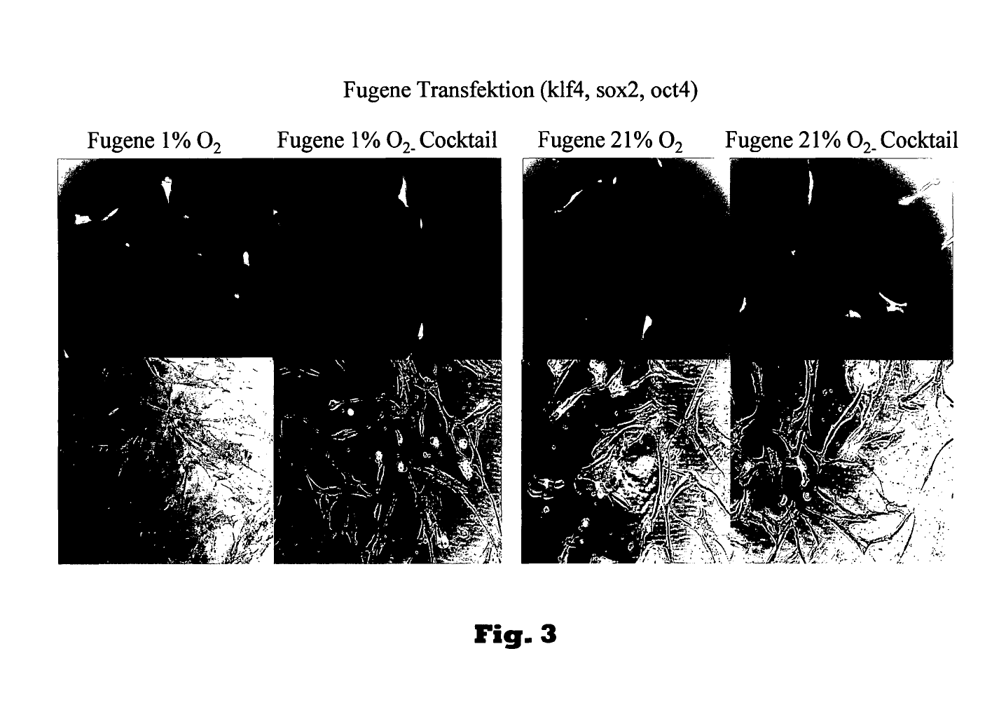

FIG. 3: Fluorescence and morphology after Fugene transfection (klf4, sox2, oct4): Human primary fibroblasts were transfected with 0.6 μg/μl mRNA per factor and 0.2 μg/μl GFP mRNA to visualize the transfection effciency. Top line are fluorescence pictures and the bottom line shows the morphology of the cells after 7 days.

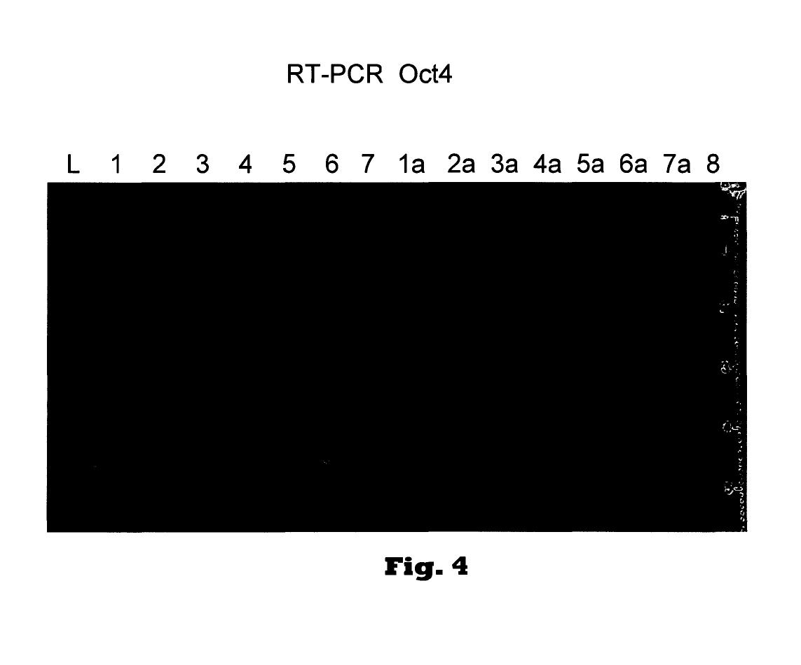

FIG. 4: RT-PCR for Oct4: L: 20 bp Ladder (Fermentas); 1 hOct4 hypoxy factors 1× transfected; 2: hOct4 hypoxy factors 6× transfected; 3: hOct4 hypoxy factors 6× transfected+Cock; 4: hOct4 normoxy factors 1× transfected; 5: hOct4 normoxy factors 6× transfected; 6: hOct4 normoxy factors 6× transfected+Cock; 7: hOct4 normoxy factors 6× transfected+Cock+MG-132; 1a: hOct4 RNA after DNase digestion; 2a: hOct4 RNA after DNase digestion; 3a: hOct4 RNA after DNase digestion; 4a: hOct4 RNA after DNase digestion; 5a: hOct4 RNA after DNase digestion; 6a: hOct4 RNA after DNase digestion; 7a: hOct4 RNA after DNase digestion; 8: Oct4 negative control.

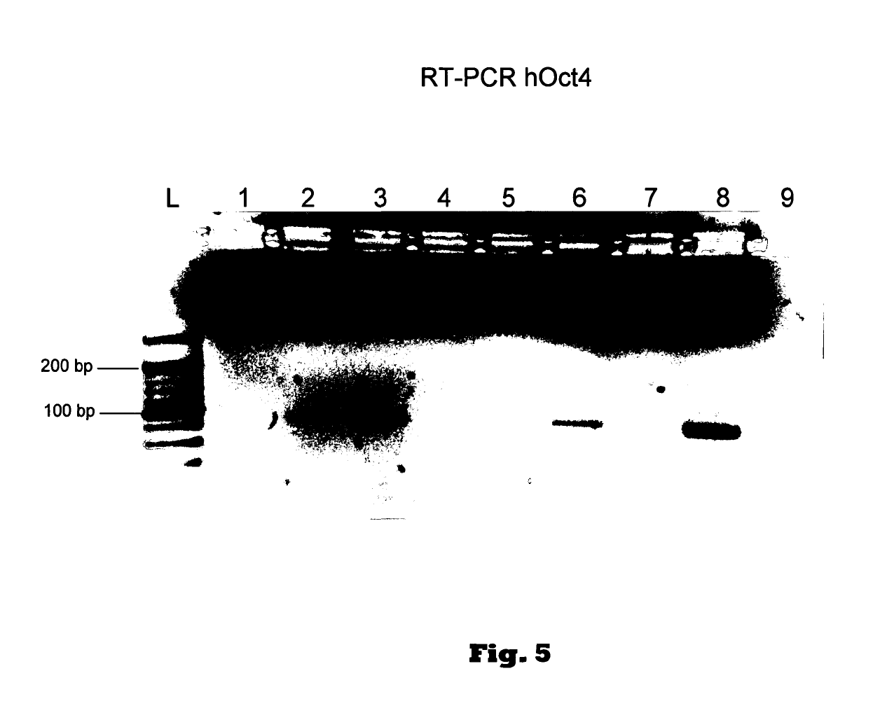

FIG. 5: RT-PCR for hOct4: L: 20 bp Ladder (Fermentas); 1: hOct4 hypoxy factors 1× transfected (human fibroblasts); 2: hOct4 hypoxy factors 6× transfected (human fibroblasts mit mRNA transfected); 3: hOct4 hypoxy factors 6× transfected+Cocktail (human fibroblasts transfected with mRNA and chemical reprogramming cocktail); 4: hOct normoxy factors 1× transfected; 5: hOct4 normoxy factors 6× transfected; 6: hOct4 normoxy factors 6× transfected+cocktail, 7: hOct4 normoxy 6× transfected factors+cocktail+MG-132; 8: hOct4 positive control (plasmid with hKlf4), 9: hOct4 negative control (water).

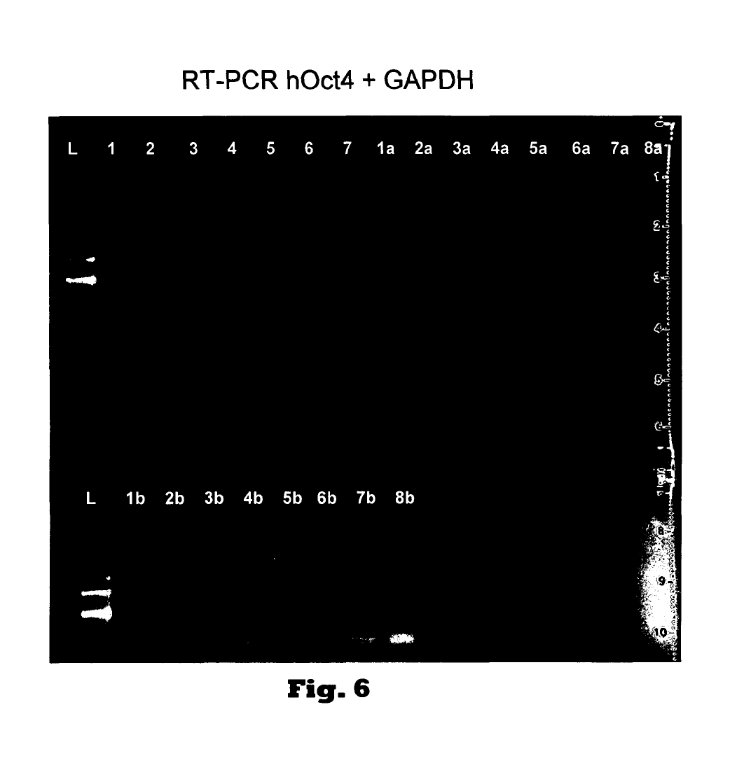

FIG. 6: RT-PCR for hOct4 and GAPDH: 1 hOct4 hypoxy factors 1× transfected; 2 hOct4 hypoxy factors 6× transfected; 3 hOct4 hypoxy factors 6× transfected+chemical cocktail; 4 hOct4 normoxy factors 1× transfected; 5 hOct4 normoxy factors 6× transfected; 6 hOct4 normoxy factors 6× transfected+chemical cocktail; 7 hOct4 normoxy factors 6× transfected+chemical cocktail+MG-132; 1a hOct4 RNA after DNase digestion; 2a hOct4 RNA after DNase digestion; 3a hOct4 RNA after DNase digestion; 4a hOct4 RNA after DNase digestion; 5a hOct4 RNA after DNase digestion; 6a hOct4 RNA after DNase digestion; 7a hOct4 RNA after DNase digestion; 8a Oct4 negative control; 1b GAPDH hypoxy factors 1× transfected; 2b GAPDH hypoxy factors 6× transfected; 3b GAPDH hypoxy factors 6× transfected+chemical cocktail; 4b GAPDH normoxy factors 1× transfected; 5b GAPDHnormoxy factors 6× transfected; 6b GAPDH normoxy factors 6× transfected+chemical cocktail; 7b GAPDH normoxy factors 6× transfected+chemical cocktail+MG-132; 8b GAPDH negative control.

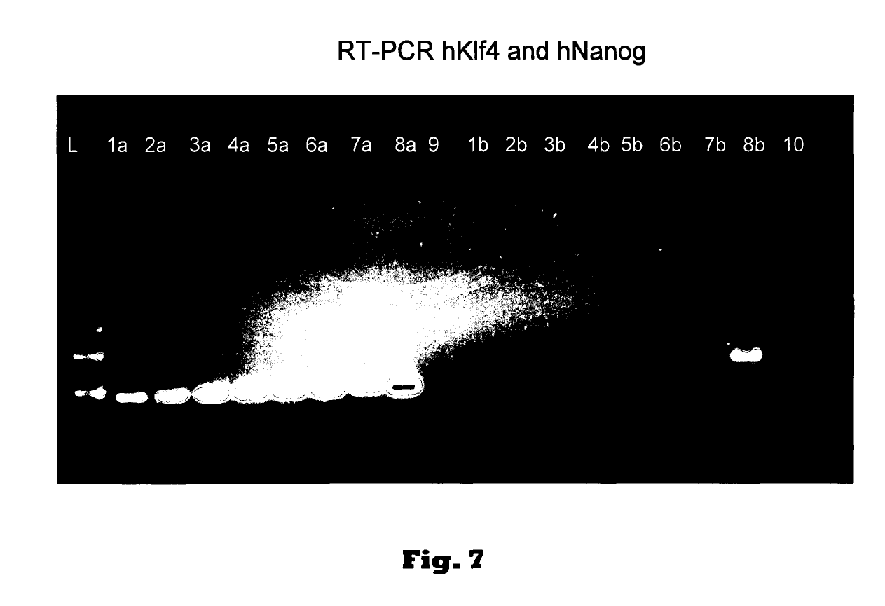

FIG. 7: RT-PCR for hKlf4 and hNanog: L: 20 bp Ladder (Fermentas); 1a: hKlf4 hypoxy factors 1× transfected (human fibroblasts); 2a: hKlf4 hypoxy factors 6× transfected (human fibroblasts with transfected mRNA); 3a: hKlf4 hypoxy factors 6× transfected+cocktail (human fibroblasts transfected with mRNA+chemical reprogramming cocktail); 4a: hKlf4 normoxy factors 1× transfected; 5a: hKlf4 normoxy factors 6× transfected; 6a: hKlf4 normoxy factors 6× transfected+cocktail, 7a: hKlf4 normoxy 6× transfected factors+cocktail+MG-132; 8a: hKlf4 pos. control (plasmid with hKlf4), 9a: hKlf4 neg. control (water) 1b: hNanog hypoxy factors 1× transfected (human fibroblasts); 2b: hNanog hypoxy factors 6× transfected (humane fibroblasts mit mRNA transfected); 3b: hNanog hypoxy factors 6× transfected+cocktail (human fibroblasts transfected with mRNA+chemical reprogramming cocktail); 4b: hNanog normoxy factors 1× transfected; 5b: hNanog normoxy factors 6× transfected; 6b: hNanog normoxy factors 6× transfected+cocktail, 7b: hNanog normoxy 6× transfected factors+cocktail+MG-132; 8b hNanog pos. control (plasmid with hNanog), 10 hNanog neg. control (water).

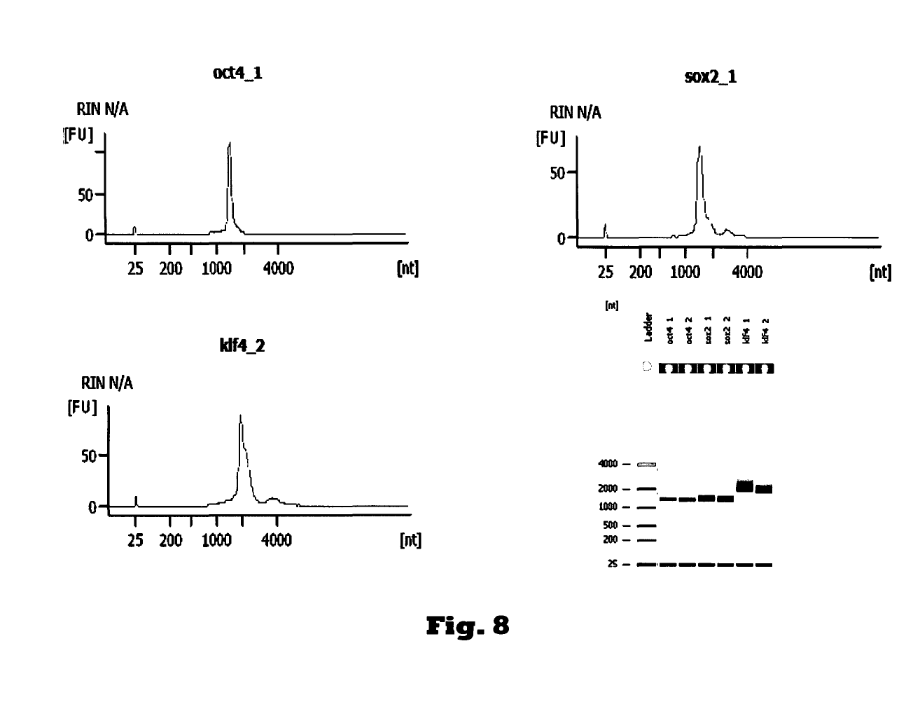

FIG. 8: Results from RNA BioAnalyzer Analysis (Agilent Nano Chip; Agilent Technologies) for oct4, sox2, and klf4:

### EXAMPLE

1. Preparation of Reprogramming mRNA

1. A) Preparation of mRNA Expression Plasmids

For the construction of plasmids comprising the target genes in a vector with the T7-promoter for in vitro transcription, using PCR, digest sites before the start-codon and after the stop codon were designed for direct cloning of the gene inserts (see Table 1) in the right direction into the pCR®II-Vector (Invitrogen).

The primers used are listed in Table 2.

TABLE 2

| | New digest sites for | | Product |
|---|---|---|---|
| Name gen | restriction enzymes | Primer sequence 5′-3′ | size |
| Nanog_human | Xbal & Notl | SEQ ID No. 17 | 918 bp |
| Nanog_human | Spel & Clal | SEQ ID No. 18 | |
| Klf4_human | Xbal & Notl | SEQ ID No. 19 | 637 bp |
| Klf4_human | Hindlll & Clal | SEQ ID No. 20 | |
| Sox2_human | Xbal & BamHl | SEQ ID No. 21 | 996 bp |
| Sox2_human | Hindlll & Clal | SEQ ID No. 22 | |
| Oct4_human | Xbal & Notl | SEQ ID No. 23 | 1100 bp |
| Oct4_human | BamHl & Clal | SEQ ID No. 24 | |
| Tert_human | Xbal & Notl | SEQ ID No. 25 | 783 bp |
| Tert_human | Hindlll & Clal | SEQ ID No. 26 | |
| GFP | Xbal & Notl | SEQ ID No. 27 | 724 bp |
| GFP | Hindlll & Clal | SEQ ID No. 28 | |

A PCR for obtaining the desired digest sites was carried out with the Platinum Taq-Polymerase (Invitrogen).

Preparation of PCR-Reaction on Ice:

| | | |
|---|---|---|
| 5 | μl | 10X PCR Buffer |
| 1 | μl | 10 mM dNTP mixture 0.2 mM each |
| 1.5 | μl | 50 mM MgCl₂ |
| 1 | μl | Primer mix (10 μM each) 0.2 μM each |
| 1 | μl | Template DNA |
| 0.2 | μl | Platinum ® Taq DNA Polymerase 1.0 unit |
| to 50 | μl | DEPC-H₂O |

PCR-Program (Biometra Tprofessional):

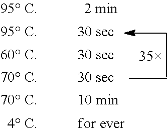

After electrophoresis in a 1% TAE-agarose-gel, specific DNA-bands (for their size see Table 2) were cut out and the DNA extracted and purified with the QIAquick Gel Extraction Kit (from Qiagen)

Procedure:
excise the DNA fragment from the agarose gel with a scalpel
weigh the gel slice
add 3 volumes of Buffer QG to 1 volume of gel (For example, add 300 μl of Buffer QG to each 100 mg of gel)
incubate at 50° C. for 10 min (or until the gel slice has completely dissolved)
add 1 gel volume of isopropanol to the sample and mix
place a QIAquick spin column in a provided 2 ml collection tube
to bind DNA, apply the sample to the QIAquick column, and centrifuge for 1 min at 13,000 rpm
discard flow-through
add 0.5 ml of Buffer QG to QIAquick column and centrifuge for 1 min at 13,000 rpm
to wash, add 0.75 ml of Buffer PE to QIAquick column and centrifuge for 1 min at 13,000 rpm
discard the flow-through
centrifuge the QIAquick column for an additional 1 min at 13,000 rpm
place QIAquick column into a 1.5 ml micro centrifuge tube to elute DNA, add 50 μl of H₂O to the centre of the QIAquick membrane, let the column stand for 1 min, and then centrifuge for 1 min at 13,000 rpm

Digest of pCRII-Vector with the specific restriction enzymes for cloning of target-DNA

Strategy for Digest of Vector pCRII:

vector pCRII digest with XbaI and SpeI for target gene human Nanog

vector pCRII digest with XbaI and HindIII for target gene human Klf4

vector pCRII digest with XbaI and BamHI for target gene human Sox2

vector pCRII digest with XbaI and BamHI for target gene human Oct4

vector pCRII digest with XbaI and HindIII for target gene human TERT

vector pCRII digest with XbaI and HindIII for target gene GFP

Preparation of Digest on Ice:

| | | |
|---|---|---|
| 5 | μl | 10x Buffer |
| ≧2 | μg | plasmid |
| 2 | μl | restriction enzyme |
| to 50 | μl | DEPC-water |

incubation for 1 h at 37° C. (thermo block; Eppendorf)
heat inactivation for 20 min by the specific temperature of the enzyme

Ligation of the target-gene inserts DNA with the linear vector DNA with the T4-DNA-Ligase (Fermentas)

Preparation of the Ligation on Ice:

| | | |
|---|---|---|
| 50-400 | ng | vector DNA |
| 50-400 | ng | insert DNA |
| 2 | μl | 10x Buffer |
| 0.2 | μl (1 u) | T4-DNA Ligase |
| to 20 | μl | DEPC-water |

vortex the tube and spin down in a micro centrifuge for 3-5 seconds
incubate on room temperature for 1 h
heat inactivation for 20 min

1. B) Transformation of the Plasmids with Chemical Competent DH5α-Bacteria
add 1 μl of the plasmids to 10 μl chemical competent DH5α-bacteria
incubation for 30 min on ice
heat shock at 42° C. for 45 sec
add 250 μl SOC-Medium to the bacteria
incubation at 37° C. for 1 h with shaking
plate out the bacteria solution on LB-kanamycin-agarose-plate and incubate the plate for 12 hours in 37° C. incubator
pick one colony of the bacteria and put the colony in 5 ml LB-medium and incubate the solution for 12 hours in a bacteria incubator with shaking

1. C) Plasmid DNA Purification with the NucleoSpin® Plasmid-KIT (Machery&Nagel)
5 ml of a saturated E. coli LB culture, pellet cells in a standard bench top micro centrifuge for 30 sec at 11,000×g.
discard the supernatant
for cell lysis add 250 μl Buffer A1. Re-suspend the cell pellet completely by pipetting up and down
add 250 μl Buffer A2
mix gently by inverting the tube 10 times and Incubate at room temperature for up to 5 min
add 300 μl Buffer A3. Mix thoroughly by inverting the tube 10 times
centrifuge for 5 min at 11,000×g at room temperature
place a NucleoSpin® Plasmid Column in a collection tube (2 ml) and load a 750 μl of the supernatant onto the column.
centrifuge for 1 min at 11,000×g.
discard flow-through and place the NucleoSpin® Plasmid Column back into the collection tube (2 ml)
for wash the membrane with DNA: Add 600 μl Buffer A4 (supplemented with ethanol).
centrifuge for 1 min at 11,000×g.
discard flow-through and place the NucleoSpin® Plasmid Column back into the collection tube (2 ml)
dry silica membrane: centrifuge for 2 min at 11,000×g and discard the collection tube (2 ml)
elute DNA: place the NucleoSpin® Plasmid Column in a 1.5 ml micro centrifuge tube and add 50 μl H₂O and incubate for 1 min at room temperature.
centrifuge for 1 min at 11,000×g
measure the DNA-concentration with the nanodrop
DNA store at −20° C.

1. D) Linearisation of the pCRII-hoct4, pCRII-hSox2, pCRII-hKlf4 Plasmids for the In Vitro Transcription
pCRII-hoct4 digest with BamHI
pCRII-hSox2 digest with HindIII
pCRII-hKlf4 digest with HindIII

Preparation of Digest on Ice:

| | | |
|---|---|---|
| 5 | μl | 10x Buffer |
| 2 | μg | Plasmid |
| 2 | μl | Restriction-Enzyme |
| to 50 | μl | DEPC-H₂O |

incubation for 1 h at 37° C. (thermo block; Eppendorf)
heat inactivation for 20 min by the specific temperature of the enzyme
heat inactivate both enzyme for 20 min at 80° C.

1. E) Purification of the Linearized pCRII-hoct4, pCRII-hSox2, pCRII-hKlf4 Plasmids with QIAquick PCR Purification Kit (from Qiagen)
add 5 volumes of Buffer PB to 1 volume of the digest sample and mix
place a QIAquick spin column in a provided 2 ml collection tube
to bind DNA, apply the sample to the QIAquick column and centrifuge for 60 s
discard flow-through
place the QIAquick column back into the same tube
to wash, add 0.75 ml Buffer PE to the QIAquick column and centrifuge for 60 s
discard flow-through and place the QIAquick column back in the same tube
to dry the membrane, centrifuge the column for an additional 1 min
place QIAquick column in a 1.5 ml micro centrifuge tube
to elute DNA, add 10 μl H₂O to the centre of the QIAquick membrane, let the column stand for 1 min, and centrifuge the column for 1 min
measure the DNA-concentration with the nanodrop
store DNA at −20° C. 1. F) In Vitro Transcription of the Linearized pCRII-hoct4, pCRII-hSox2, pCRII-hKlf4 Plasmids with mMessage mMachine High Yield Capped RNA Transcription Kit (from Ambion) and Add a Poly(A) Tail to RNA Transcripts (Poly(A)-Tailing Kit from Ambion)

Preparation of the In Vitro Transcription (mMESSAGE mMACHINE Reaction):

| to 20 | μl | nuclease-free water |
|---|---|---|
| 10 | μl | 2x NTP/CAP |
| 2 | μl | 10x reaction buffer |
| 1 | μg | linear template plasmid |
| 2 | μl | enzyme mix |

pipette the mixture up and down gently
short centrifugation to collect the reaction mixture at the bottom of the tube
incubation for 2 h at 37° C.
add 1 μl TURBO DNase to the mRNA and mix well
incubate at 37° C. for 15 min
direct after the DNase-treated reaction add the poly(A)-tail to the mRNA

Preparation for Poly(A)-Tail Reaction:

| | |
|---|---|
| 20 μl | mMESSAGE mMACHINE reaction |
| 36 μl | nuclease-free water |
| 20 μl | 5x E-PAP Buffer |
| 10 μl | 25 mM MnCl₂ |
| 10 μl | mM ATP |
| 4 μl | E-PAP |

mix gently
incubate at 37° C. for 1 h
place reaction on ice
lithium chloride precipitation of the mRNA with poly(A)-tail:
add 30 μl LiCl Preciptitation Solution
mix thoroughly
chill for 30 min at −20° C.
centrifuge at 4° C. for 15 min at maximum speed to pellet the RNA
remove the supernatant
wash the pellet once with 1 ml 70% ethanol
centrifuge at 4° C. for 15 min at maximum speed to pellet the RNA
remove the 70% ethanol
air-dry the pellet
solve the RNA-Pellet with 30-50 μl nuclease-free-water
the concentration of the RNA is measured with the nanodrop
the quality of the RNA is measured with the Agilent nano-chip

2. Preparation of Target Cells

2. A) Preparation of Mouse and Rat MSCs
kill the rat or mouse with CO₂-gassing
spray the rats and mice with 70% Ethanol to kill of bacteria and fungal spores
remove the coat from the skin
remove the back legs cleanly at the hip joint
under sterile conditions, remove all soft tissue and separate the bones
remove the growth plates both femur and tibia
insert holes with a needle into the bones on both ends
place the bones in eppendorf tubes (for mice) or in falkon tubes (for rats) and spin at 2000 rpm for 1 min (all of the bone marrow will be deposited in the bottom of the tube sitting in spacers); remove the bones and spacer
re-suspend the marrow in 0.5 ml medium (DMEM low glucose; 10% FCS; 1% Pen/Strep)
disseminate the marrow of one leg from a rat in 1 T-75 flask with 12 ml medium
disseminate the marrow of two legs from a mouse in 1 10 cm petri-dish with 12 ml medium

2. B) Preparation of Human MSC
get a bone marrow aspirate
re-suspend in DMEM (low glucose, 10% FCS)
plate out in a tissue culture flask
growth for 5 days and then change media
thereafter change the medium twice weekly
culture in tissue culture flask until 80% confluency and subculture until needed

2. C) Preparation of Mouse and Rat Fibroblasts
cut of the tail
shave the tail with a scalpel
spray the tail with 70% ethanol to kill of bacteria and fungal spores
remove the skin from the tail
place the skin in a 10 cm petri dish
under sterile conditions, cut the skin in small pieces (5×5 mm)
digest the skin-pieces with 0.05% Trypsin EDTA for 20 min in 37° C. CO₂-Incubator
wash the trypsin from the skin-dishes with medium (DMEM high glucose, 10% FCS, 1% Pen/Strep), remove the medium and wash again for 3-4 times
then incubate the skin pieces in 37° C. CO₂-Incubator and change the medium every day, after 1 week the fibroblasts growth out

2. D) Preparation of Human Fibroblasts
place prepuce in petri dish
wash two times with PBS
remove fat
wash with PBS one time
cut the skin in small pieces (2 mm)
add 10 ml dispase (2 U/ml)
incubate at 4° C. for 16-18 h
discard dispase solution
add 5 ml PBS
seperate dermis and epidermis
transfer dermis to new dish and add 10 ml collagenease (500 U/ml)
make very small pieces
transfer pieces into a 50 ml falcon tube with solution and incubate for 45 min (37° C.; 5% CO₂)
centrifuge at 1000 rpm for 5 min
discard supernatant and resuspend pellet in DMEM (10% FCS)
centrifuge again
resuspend pellet in 2 ml DMEM (10% FCS) and transfer into a T75 cell culture flacon
expanded cells until use

3. mRNA Transfection

3. A) mRNA-Transfection of Human Fibroblasts, Human MSC, Rat Fibroblasts, Rat MSC, Mouse Fibroblasts and Mouse MSC with AMAXA, FugeneHD, Lipofectamine™ LTX Reagent and PLUS™ Reagent

1) AMAXA-Transfection with the Human Dermal Fibroblast Nucleofector® Kit (6 Well-Plate):

Preparation for One Electroporation in Certified Amaxa Cuvette:
mRNA of oct4, sox2 and klf4 in sum of 2 μg
4×10⁵ cells
100 μl Nucleofector® Solution
choose the program U-023 by the Amaxa-electroporation-machine
re-suspend the cells in the cuvette with 500 μl fibroblast or MSC-Medium
provide 5 ml fibroblast or MSC-Medium with or without Cock tail in every well of a 6 well-plate
plate out 100,000 cells in each well of a 6 well-plate

Cocktail Composition:

| | | |
|---|---|---|
| Resveratrol | 0.05 | mM |
| Selenium | 0.1 | μM |
| EGCG ((—)-Epigallocatechin gallate) | 0.01 | μM |
| TSA (Trichostatin A) | 0.015 | μM |
| Reversin | 1 | μM |
| VPA (2-Propylpentanoic acid free acid) | 6 | μM |
| 5′Aza (5-Aza-2′-deoxycytidine) | 1 | μM |

at day 1: the cocktail is with 5′Aza and without TSA; after 48 hours 5′Aza replaced by TSA
every 72 hours new transfection with FuGENE® HD Transfection Reagent (Roche)

2) Transfection FuGENE® HD Transfection Reagent (Roche)
before transfection: change to antibiotic free medium

Transfection Procedure
Dilute mRNA with serum-free and antibiotic-free medium to a concentration of 2 μg mRNA at a volume of 100 μl for each well (6 well-plate)
Pipet the 8 μl FuGENE® HD Transfection Reagent (Ratio: 8:2 Transfection reagent to mRNA) directly into the medium pipette up and down
Incubate the transfection reagent: mRNA complex for 15 minutes at room temperature
Add the transfection complex to the cells in a drop-wise manner
Swirl the wells to ensure distribution over the entire plate surface
after 4-6 hours change to medium (contains 10% FCS, 1% Pen/Strep) with or without cocktail
one transfected 6 well plate incubate at 21% O₂-condition (normoxy) and a second plate incubate at 1% O₂-condition (hypoxy) at 37° C. incubator

3) Alternative Transfection with Lipofectamine™ LTX Reagent or PLUS™ Reagent (Invitrogen)

a) Transfection FuGENE® HD Transfection Reagent (Roche)
plate out 7000 cells in 500 μl medium per well of a 24 well-plate
incubate over night
for transfection: change to antibiotic free medium
dilute 0.5 μg mRNA in 25 μl serum-free and antibiotic-free medium for each well (24 well-plate)
pipette the FuGENE® HD Transfection Reagent 2 μl (Ratio: 8:2 Transfection reagent to mRNA) directly into the medium
pipette up and down
incubate the transfection reagent: mRNA complex for 15 minutes at room temperature
add the transfection complex to the cells in a drop-wise manner
swirl the wells to ensure distribution over the entire plate surface
after 4-6 hours change to medium (contains 10% FCS, 1% Pen/Strep) with or without cocktail

b) Lipofectamine™ LTX Reagent and PLUS™ Reagent (Invitrogen)
plate out 7000 cells in 500 μl Medium per well of a 24 well-plate
incubation over night
before transfection: change to antibiotic free medium
dilute 0.5 μg mRNA in 100 μl serum-free and antibiotic-free medium for each well (24 well-plate)
mix gently
mix PLUS™ Reagent gently before use
add 0.5 μl PLUS™ Reagent in the mRNA-Medium mix, mix gently and incubate 5 min at room temperature mix Lipofectamine™ LTX Reagent gently before use
add 1.25 μl directly to the diluted mRNA; mix gently
incubate for 30 min at room temperature
add the 100 μl mRNA-Lipofectamine™ LTX Reagent complex to the well
mix gently by rocking the plate back and forth
incubate the cells at 37° C. in the CO₂ incubator for 4-6 hours
change to medium with or without cocktail
during the optimization of the transfection efficiency→in crease of the amount of mRNA concentration at 1 μg per well (24 well plate)
every 72 h repeat the transfection with FuGENE HD or Li pofectamine™ LTX Reagent and PLUS™ Reagent

4. Protease Inhibitor Treatment (MG132) to Increase the Half-Life of the Proteins Produced by the Transfected mRNA:
48 h after transfection: treatment of two wells in every plate with MG-132 (10 μM/well) with and without cocktail for 6 h, than change medium

5. Results

Analysis of mRNA amount (per PCR) of the transfected or internal genes (oct4, nanog, hTERT, sox2, klf4) and promoter analysation (of nanog, sox, oct4)

The reprogramming results are set forth in FIGS. 1 to 8

6. Tools and Methods

6.1 RNA-Isolation with TriFast™ (Peglab)
1 ml Trifast per 10 cm2 of the culture dish area
samples can be stored for a long time at −80° C. or be directly used
samples should be kept for 5 minutes at room temperature
addition of 0.2 ml of chloroform
shake samples by hand vigorously for 15 seconds
incubation for 3 minutes at room temperature
centrifugation at 12,000×g
transfer the aqueous phase with RNA to new tube (save the interphase and organic phase at 4° C. for the isolation of DNA and proteins).
precipitation of the RNA with 0.5 ml of Isopropanol per 1 ml of Tri-Fast™ used for the initial homogenization.
incubation on ice for 15 min
centrifuge for 10 minutes at 4° C. at 12,000 g
remove the supernatant
wash the RNA pellet twice with 75% ethanol by vortexing
subsequent centrifugation for 8 minutes at 7,500×g (4° C.)
remove the excess isopropanol from the RNA pellet by air-drying
re-suspend the RNA pellet in RNase-free water (DEPC-H₂O)→30 μl-50 μl
dissolve the RNA pellet by passing the solution through a pipette tip several times
incubating the solution for 10 min at 55° C.
pipetting it up and down
heating the sample at 55-60° C. might help to dissolve the pellet
measure the RNA-Concentration→NanoDrop
samples store at −80° C. for 2-3 months

6.2 DNA-Isolation
Remove any left aqueous phase after RNA removal
precipitate the DNA with 0.3 ml of 100% ethanol per ml Tri-Fast™
mix well by inversion
store the samples at room temperature for 2-3-minutes
centrifugation at 2,000×g at 4° C. for 5 minutes
remove the DNA supernatant (store it at 4° C. for the protein isolation)
DNA pellet washed twice with 1 ml 0.1 M sodium citrate in 10% ethanol
At each wash step keep the DNA in 0.1 M sodium citrate/10% ethanol for 30 minutes at room temperature (with periodic mixing)
centrifuge at 4° C. for 5 minutes at 2,000×g
after these two washes suspend the DNA pellet in 2 ml of 75% ethanol
keep it at room temperature with periodic mixing for 15 minutes
centrifuge at 2,000×g for 5 minutes at 4° C.
dry the DNA pellet briefly for 5-10 minutes under vacuum and
dissolve it in 8 mM NaOH by slowly passing the pellet through a wide bore pipet.
adjust the final concentration of DNA to 0.2-0.3 μg/μl with 8 mM NaOH

6.3 RNA Treatment with DNase I (Fermentas):

| | | |
|---|---|---|
| 1 | μg | RNA |
| 1 | μl | 10X reaction buffer with MgCl₂ |
| 1 | μl (1 u) | DNase |
| to 9 | μl | DEPC-H₂O |

incubate at 37° C. for 30 minutes
inactivation of DNase I: Add 1 μl 25 mM EDTA and incubate at 65° C. for 10 min

6.4 RT-PCR with SuperScript™ III Reverse Transcriptase (Invitropen)

Reaction Condition:

| | | |
|---|---|---|
| 1 | μl | oligo(dT)20 Primer |
| 1 | μg | RNA |
| 1 | μl | 10 mM dNTP Mix (10 mM each dATP, |
| | | dGTP, dCTP and dTTP) |
| to 13 | μl | DEPC-H₂O |

heat mixture to 65° C. for 5 minutes
incubate on ice for 1 min

Add:

| | |
|---|---|
| 4 μl | 5X First-Strand Buffer |
| 1 μl | 0.1 M DTT |
| 1 μl | SuperScript ™ III RT (200 units/μl) |

mix by pipetting gently up and down
incubate at 50° C. for 60 min
inactivate the reaction by heating at 70° C. for 15 minutes
store the cDNA at −20° C.

6.5 PCR of Human oct4 (Control the Human Fibroblasts):

Primersequence: hoct4_s: gaggatcaccctgggatataca
hoct4_as: agatggtcgtttggctgaatac

Product size: 100 bp

6.6 PCR with Patinum Taq-Polymerase (Invitroqen)

Preparation of PCR-Reaction on Ice:

| | | |
|---|---|---|
| 5 | μl | 10X PCR Buffer |
| 1 | μl | 10 mM dNTP mixture 0.2 mM each |
| 1.5 | μl | 50 mM MgCl₂ |
| 1 | μl | Primer mix (10 μM each) 0.2 μM each |
| 1 | μl | Template DNA |
| 0.2 | μl | Platinum ® Taq DNA Polymerase 1.0 unit |
| to 50 | μl | DEPC-H₂O |

PCR-Program (Biometra Tprofessional):

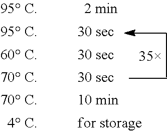

PCR-control with 3% TAE-agarose-gel for electrophoresis

6.7 Protocol to Produce iPScells

6.7.1 BD Matrigel™ hESC-Qualified Matrix
put the sterilized tip box and eppendorf tubes over night in −20° C. freezer
thaw the Matrigel bottle on ice in the 4° C. fridge overnight
aliquot Matrigel for hESC upon arrival, store at −80° C., only thaw once and do not re-freeze to avoid breakdown of growth factors
for coating purposes, dilute Matrigel appropriately by using serum-free medium (0.3 μg/μl for iPS, 1 μg/μl for hESC)
add 50 μl Matrigel-dilution per cm² growth surface
incubate 60 min at RT under sterile conditions or overnight at 4° C.
soak off remaining Matrigel/medium
Medium: mTeSR1 (Stem Cell Technology)

The coating recipe (0.3 ug/μl) is dependant on the cell type that is used to generate iPS. It is recommended to coat and not to gel plastics for hESC culture using 1 ug Matrigel/μl. Use plates immediately or store for max 7 days at 4° C. covered with serum-free medium under aseptical conditions (sealed).

The coated Matrigel plates should always be processed, stored and used exactly the same way to avoid variabilities (mainly through breakdown of growth factors). In the beginning or for establishment it is necessary to titrate a coating volume and—concentration (for instance 3 dilutions 0.3, 0.6, 1 ug/ul) that suit the specific type of cells best.

6.7.2 BD Matrigel™ Basement Membrane Matrix, Growth Factor Reduced (GFR)
aliquote the matrigel such as hESC-qualified Matrigel
thaw tube overnight on ice at 4° C.
dilute with 6 ml cold basal media and mix well
add 1 ml per well of 6 well plate
incubate the plate at room temperature for one hour or over-night at 4° C.
plate may either be used immediately or stored at 4° C. (plate are be good for least one week)
remove the excess liquid and wash once with basal medium
basal medium component: D-MEM/F-12; 20% KO Serum Replacer; 1% Non-essential Amino Acids; 1 mM L-Glutamine, 0.1 mM 2-Mercaptoethanol; 100 ng/ml bFGF
use mouse cells then add LIF-supernatent to the basal medium

6.7.3 Production of LIF-Supernatant
coat petri dish (145 mm) with 0.1% gelantine (Bovine Type x?, Sigma)
plate 3×10 e10 LIF producing feeder cells SNL in every dish
Medium component: D-MEM high glucose, 10% FCS, 1% Pen/Strep; 1% L-Glutamine
after 24 hours collect the medium from the dish and filter through a 0.22 μm filter
aliquote the medium containing LIF in 500 μl aliquots and store at −20° C.
change media after 24 hours or 48 hours

1. A process for preparing reprogrammed cells in vitro, comprising:
a) providing cells to be reprogrammed;
b) introducing reprogramming mRNA molecules into the cells provided in step a), wherein the reprogramming mRNA molecules are encoding at least one reprogramming protein selected from the group consisting of Ronin, Oct4, Klf4, Sox2, Nanog and TERT;
c) culturing the cells obtained in step b) in a cell culture medium and under a condition suitable to allow translation of the transfected reprogramming mRNA molecules so as to obtain reprogrammed cells.

2. The process according to claim 1, wherein the condition suitable to allow translation is an oxygen content in the cell culture medium from 0.5% to 21%.

3. The process according to claim 1, wherein the condition suitable to allow translation is a temperature from 30° C. to 38° C.

4. The process according to claim 1, wherein step c) includes culturing the cells obtained in step b) in a cell culture medium having a glucose content of 0.1 g/l to 4.6 g/l.

5. The process according to claim 1, wherein step c) includes culturing the cells obtained in step b) in a cell culture medium containing at least one inducing substance selected from the group consisting of reversin, resveratrol, selenium, selenium containing compounds, EGCG ((−)-epigallocatechin-3-gallate), valproic acid, salts of valproic acid and sodium valproate.

6. The process according to claim 1, wherein step c) includes culturing the cells obtained in step b) in a cell culture medium containing at least one transient proteolysis inhibitor.

7. The process according to claim 6, wherein the transient proteolysis inhibitor is selected from the group consisting of protease inhibitor, proteasome inhibitor and lysosome inhibitor.

8. The process according to claim 1, wherein the cells to be reprogrammed are human cells.

9. The process according to claim 1, wherein the cells to be reprogrammed are non-embryonic stem cells.

10. A process for the production of cells exhibiting a pluripotent or multipotent stem cell character, wherein a process according to claim 1 is carried out.

11. A process for inducing the dedifferentiation of cells, wherein a process according to claim 1 is carried out.

12. A process for improving the expansion of non-embryonic stem cells in vitro, wherein a process according to claim 1 is carried out.

13. A process for the rejuvenation of aged cells, wherein a process according to claim 1 is carried out.

14. A multipotent or pluripotent stem cell prepared according to claim 1.

15. A process for reprogramming recipient cells of a mammal in vivo comprising: introducing reprogramming mRNA molecules into the recipient cells of the mammal, wherein the reprogramming mRNA molecules are encoding at least one reprogramming protein selected form the group consisting of Ronin, Oct4, Klf4, Sox2, Nanog and TERT.


SEQUENCE LISTING (30 sequences)

SEQ ID NO: 1 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 2 - DNA, 1356 bp, Mus musculus
tctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgg

SEQ ID NO: 3 - DNA, 2358 bp, Rattus norvegicus
tcagataggctgatttcgagtctttctcttttgtgggaagaccgaggctcgcttctttttggcttgttgactcttttacatctggacatttaactcttacttttaagatctttccctctagacactgagttttaaagtcttaactttttggttgttaaaaacttttttttttttaaagtcccttcccttgccgttgggctgacatgagcgtggatctttctggtccccacagtctgcctagttgtgaggaagcatcgaactctggggattcctcgccgatgcctgccgttcatcttcctgaggaaaattattcttgcttacaagtgtctgctactgagatgctctgcacagagactgcctctcctccgccttcctctggggacctacctcttcaagatagccctgattcttctagcaatcccaagctaaagctgtctggtcccgaggctgacgagggccctgagaagaaagaagagaacaaggtcctcaccaagaagcagaagatgcggactgtgttctctcaggcccagttgtgtgcactcaaggataggtttcagaggcaaaggtacctcagcctccagcagatgcaagatctctctaccattctgaacctgagctataagcaggtgaagacctggttccaaaaccaaagaatgaagtgcaagaggtggcagaaaaaccaatggttgaagactagcaacggcctgactcagaagggctcagcgccggtggagtatcccagcatccattgcagctattctcagggctatctgatgaacgcgtctggaaaccttccagtatggggcagtcagacctggaccaacccaacttggaacaaccagacctggaccaacccaacctggagcaaccagacctggaccaacccaacttggagcaaccaggcctggagcactcagtcctggtgtactcaggcctggaacagccagacttggaacgctgctccgctccataacttcggggaggactccctgcagccttatgtgccgttgcagcaaaacttctccgccagtgatttggaggcgaatttggaagccactagggaaagccaggcgcattttagtaccccgcaagccttggaattgttcctgaactactccgtgaattctccaggcgaaatatgaggtttacacaacaactgggcttaaagtcagggcagggccagggtcagctttcttccttcttccaaagagttttatattgttcttattttttttttaattattattttgtttttgttttttgtttatcaaggtagggtttctctgtgtggttctggctgtcctggaattcactctgtagaccaggctggcctcgtactcagagatctgcctacttttgcctcctgaaggctagggctaaagattttctaaagattttcatagtttttatttttttaattattatctgttttcatgtttgtgtttttttgttttgtttttgtttttgtttttgtttatcaagatagggtttctctgtgtggctctagtagtcccggaaactggctctgtagaccaggctgtccttgaactcagaaatctgcctttgcctccggactgcggggactaaaggcagtatataaccacctggcacattgtttttatttttattcttttggtgccagaaagcaaacctaggactttgagctgggcacccactcaaccactgagctctgtttgcgacccccgtgttggctgcatttgtctgagctgggtaacttgtctttttttccgtgttaacgatgggcttcggagacagtgcactatacactctatcctcccccaggtctcacacacccaccctactccataccaacccaggcttgtctgtcttttttttttttggagctgaggactgaacccagggccttgcgctttctaggcaagcgctctacctctgagctaaatccccaacccttgtctgtctttttagaagcttgggtcttggtgtgcactgtgtatcgttttgaggggtgaggtttaaaagtatacaaattataaagattcatgcagatatggtggctcctctcaaggacgagacagaaggatcaccagtttgaggctatctcagatataaaataagttcaagaccagcctgtactatgtctaaatagtaagacagcatctcaacaaaataataaaactaaggtaaggagataaaagtaaagtctcaacaaaatacaagatctcgcctgttacagttctttgatttcctccgtgtctttgcagttccgccaagaggcttctatgttaatatctgtagaaagatgtttatatttgactgtaccatgataaaccagtgccagctggactagtttaaataaaacactaattttaccca

SEQ ID NO: 4 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 5 - DNA, 2457 bp, Mus musculus
ctattaacttgttcaaaaaagtatcaggagttgtcaaggcagagaagagagtgtttgcaaaaagggaaaagtactttgctgcctctttaagactagggctgggagaaagaagaggagagagaaagaaaggagagaagtttggagcccgaggcttaagcctttccaaaaactaatcacaacaatcgcggcggcccgaggaggagagcgcctgttttttcatcccaattgcacttcgcccgtctcgagctccgcttccccccaactattctccgccagatctccgcgcagggccgtgcacgccgaggcccccgcccgcggcccctgcatcccggcccccgagcgcggcccccacagtcccggccgggccgagggttggcggccgccggcgggccgcgcccgcccagcgcccgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggactgcgaactggagaaggggagagattttcaaagagatacaagggaattgggaggggtgcaaaaagaggagagtaggaaaaatctgataatgctcaaaaggaaaaaaaatctccgcagcgaaacgacagctgcggaaaaaaaccaccaatcccatccaaattaacgcaaaaaccgtgatgccgactagaaaacttttatgagagatcttgggacttctttttgggggactatttttgtacagagaaaacctgagggcggcggggagggcgggggaatcggaccatgtatagatctggaggaaaaaaactacgcaaaacttttttttaaagttctagtggtacgttaggcgcttcgcagggagttcgcaaaagtctttaccagtaatatttagagctagactccgggcgatgaaaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaatttaggaccgttacaaacaaggaaggagtttattcggatttgaacattttagttttaaaattgtacaaaaggaaaacatgagagcaagtactggcaagaccgttttcgtggtcttgtttaaggcaaacgttctagattgtactaaatttttaacttactgttaaaggcaaaaaaaaaatgtccatgcaggttgatatcgttggtaatttataatagcttttgttcaatcctaccctttcattttgttcacataaaaaatatggaattactgtgtttgaaatattttcttatggtttgtaatatttctgtaaattgtgatattttaaggtttttccccccttttattttccgtagttgtattttaaaagattcggctctgttattggaatcaggctgccgagaatccatgtatatatttgaactaataccatccttataacagctacattttcaacttaagtttttactccattatgcacagtttgagataaataaatttttgaaatatggacactgaaa

SEQ ID NO: 6 - DNA, 2323 bp, Rattus norvegicus
gtgtttgcaaaaagggaaaagtactttgctgcctctttaagactagggctgggagaaagaagaggagagaaaaagaaaggagagaagtttggagcccgaggcttaagcctttccaaaaactaatcacaacaatcgcggcggcccgaggaggagagcgactgttttttcatcccaattgcacttcgcccgtctcgagctccgcttccccccaactattctccgccagatctccgcgcaaggccgtgcacgccgacgaccccgcccgcggcccctgcatcccggcccccgcgcgcggcccccgcagtcccggccgggccgagggtcggcggccgccggcgggccgcgcccgcgcccagcgcccgcatgtataacatgatggagacggagctgaagccgccgggccctcagcaagcttcggggggcggcggcggaggaggcaacgccacggcggcggcgaccggcggcaaccagaagaacagcccggaccgcgtcaagaggcccatgaatgccttcatggtgtggtcccgggggcagcggcgtaagatggcccaggagaaccccaagatgcacaactcggagatcagcaagcgcctgggcgccgagtggaaacttttgtcggagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcagccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagttccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggccggaccgcgaactggagaaggggagagatttttcaaaaagatacaagggaattgggaggggtgcaaaagaggagagtaagaaaaatctgaatgctcaaaaggaaaaaaaaaatctcattacccgcagcaaaatgacagctgcggaaaaaaaccaccaatcccatccaaattaacgcaaaaaccgtgatgccgactagaaaacttttatgagagatctggaggaaaaaaactacgcaaaacttttttttaaagttctagtggtacgttaggcgcttcgcagggagttctcaaaagtctttaccagtaatatttagaactagactccgggcgatgaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaattaaggaccgttacagacaaggaaggaatttattcggatttgaacgttttagttttaaaattgtacaaaaggaaaacatgagagcaagtactggcaagaccattttcgtggtcttgtttagggcaaacgttctagattgtactaaatttttaacttactgttaaaggcaaaaaaaaaatgtccatgcaggttgatatcgttggtaatttataatagcttttgttcaatcccacccttttcattttgttcacataaaaatatggaaattactgtgtttgaaatattttcttatggtttgtaatatttctgtaaattgtgatattttaaggttttttcccccttttattttccgtagttgtattttaaaagattcggctgttattggaaccaggctgccgagaatccatgtatatatttgaactaataccatccttataacagttacgtttccaacttaagtttttactccattatgcacagtttgagataaataaatttttgaaatatggacactga

SEQ ID NO: 7 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 8 - DNA, 1346 bp, Mus musculus
aaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagct

SEQ ID NO: 9 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 10 - DNA, 3057 bp, Mus musculus
agttccccggccaagagagcgagcgcggctccgggcgcgcggggagcagaggcggtggcgggcggcggcggcacccggagccgccgagtgcccctccccgcccctccagccccccacccagcaacccgcccgtgacccgcgcccatggccgcgcgcacccggcacagtccccaggactccgcaccccgcgccaccgcccagctcgcagttccgcgccaccgcggccattctcacctggcggcgccgcccgcccaccgcccggaccacagcccccgcgccgccgacagccacagtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaagaaggatctcgggcaatctgggggttttggtttgaggttttgtttctaaagtttttaatcttcgttgactttggggctcaggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagttcaagagacaaaacagaaatactaaaaacaaacaaacaaaaaaacaaacaaaaaaaacaagaaaaaaaaatcacagaacagatggggtctgatactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttgtatgctctttgccttcttaaaaaaaaaaaaagccattattgtgtcggaggaagaggaagcgattcaggtacagaacatgttctaacagcctaaatgatggtgcttggtgagtcgtggttctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaacgacaaaaaaaaaaaaaaa

SEQ ID NO: 11 - DNA, 2393 bp, Rattus norvegicus
atcttcgttgacttcggggtttgggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgagaggaactctctcacatgaagcgacttcccccacttcccggccgcccctacgacctggcggcgacggtggccacagacctggaaagtggtggagctggtgcagcttgcagcagtaacaacccggccctaccccggagggagaccgaggagttcaacgatctcctggacctagactttatcctttccaactcgctatcccaccaggaatcggtggccgccaccgtgaccacctcggcgtcagcttcatcctcgtcttccccagctagcagcggccctgccagcgcgccctccacctgcagcttcagctatccgatccgggccgggggtgacccgggcgtggctgcgggcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgagcacccctggcagcgagtacaccagcccttcggtcatcagtgttagcaaaggaagcccagacggcagccaccctgtggtagtggcgccctacagcggtggcccgccgcgtatgtgccccaagattaagcaagaggccgtcccgtcctgcacggtcagccggtccctagaggcccacttgagcgctggaccccagctcagcaacggccacaggcccaacacacacgacttccccctggggcggcagctccccaccaggactacccctacactgagtcccgaggaactgctgaacagcagggactgtcaccctggcctgcctcttcccccaggattccatccccatccggggcccagctaccctcctttcctgccagaccagatgcagtcgcaagtcccctctctccattatcaagagctcatgccaccgggatcctgcctgccagaggagcccaagccaaagaggggaagaaggtcttggccccggaaaagaacagccacccacacttgtgactatgcaggctgtggcaaaacctatacgaagagttctcatctcaaggcacacctgcgaactcacacaggcgagaaaccttaccactgtgactgggacggctgtgggtggaaattcgcccgctcagatgaactgaccaggcactaccgcaaacacaccgggcaccggccctttcagtgccagaagtgcgacagggccttttccaggtcggaccaccttgccttacacatgaagaggcacttttaaattccacatcgtggacatgacccacactgccaggagagagttcagtatttttttttaacctttcacactgtcttcccacgaggggaggagcccagctggcaagcgctacaatcatggtcaagttcccagcaagtcagcttgtgaatggataatcaggagaaaggaagagtccaagggacaaaagaaaagaaaagaaaaaaatactaaaaaacaaacaaacaaaaaaaaaaaacaaaagaaaaaaatcacagaacagatggggtctgagactggatcttctatcattccaataccaaatccgacttgaacaagactggacttacaaaatgccaaggggtgactggaagtttgtggatatcagggtatacattaaatcagtgacctggggggagggaagaccagagttcccttgaattgtgcttcaatgatgcaatatacatggaaagaccaccttgtatgctctttgccttctaaaaagccattatgacgtcagaggaagaggaagcaattcaggtacagaacgtgttctaatagcctaaacgatggtgcttggtgagtcgtggttctaaaggtaccaaacgggggagccaaagttctccaactgctgcatactttgacaaggaaaatctatttttgtcttccgatctacatttatgacctaagtcaggtaaataagcctggtttatttctgtaacattttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaaaaaaaaaaaaaaaaaaaaaaaaaaa

SEQ ID NO: 12 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 13 - DNA, 4237 bp, Mus musculus
ggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatgg

SEQ ID NO: 14 - DNA, 3378 bp, Rattus norvegicus
atgccccgcgctcctcgttgccccgccgtgcgctctctactgcgcagccgatatcgggaggtgtggccgctggcgacctttgtgcggcgcctggggcttgagggcagtcggcttgtgcaacccggggacccgaaggtcttccgcacgttggttgcccagtgcctagtgtgcgtgccctggggctcacagccgccacctgctgacctttccttccaccaggtgtcatccctgaaagagctggtgtccagggttgtgcagaaactttgcgagcgcggtgagaggaatgtgctggcttttggctttgcactgcttaacggggccagaggtgggcctcccatggccttcacgaccagcgtgcatagctacttgcccaactcggttactgagtccctgtgtgtcagtggtgcatggatgctactgttgagccgagtgggcgacgacctgctggtctacctgctgtcgcactgtgcgctctacctgctggtgccccccagctgtgcctaccaggtgtgcgggtcacccctgtaccaaatttgtgccaccacggatacctggtcctctgtgcccgctggttacaggcccactcgacccgtgggcgggaatttcactaaccttgggtccgcacaccagatcaaaaacagtggtcaccaggaagcaccaaaaccccaggccttgccatcacgaggtacgaagaggcttctgagtctcaccagtacaaacgtgccttcagctaagaaggccaggtttgaacctgccctgagagtggataagggaccccacaggcaggtggtaccaaccccatcaggcaaaacatgggcgccaagtcctgctgcgtcccccaaggtgcctcctgcagcgaaaaacttgtctttgaaaggaaaggcatctgacccgagtctctctgggtcggtgtgctgtaaacacaagcccagctcctcgtccctgctgtcatcaccaccccaagatgctgaaaagctcaggccattcactgagaccagacatttcctttactccaggggaggtggccaagaggagctaaatccctcattcctactcaacagcctcccgcctagcttgaccggggccaggagactggtggagatcatctttctgggctcaaggcctaggacatcaggaccattctgcaggacccgccgcctgccccgtcgatactggcagatgcgacccctattccagcagctgctcatgaaccacgcaaagtgccaatatgtcagattcctccggtcgcactgcagatttcgaacagcaaaccagcgggtgccggatgccatggacaccagcccatcccacctcacgagtttgctccggttacacagcagcccctggcaggtatacggctttcttcgggcctgcctccgcgagctggtgcctgccggtctctggggcaccaggcacaatgagcgccgcttcttaaagaacgtgaagaagttcatctcgttggggaagtacgccaagctatccctgcaggaactgatgtggagggtgaaagtggaggactgccactggctccgcagcagcccagagaaggacactgtccctgccgcagagcaccgtctgagggagaggatccttgccatgttcctgttctggctaatggacacatatgtggtacagctgctgaggtcattcttctacatcacagagaccacgttccagaagaaccgccttttcttctaccgtaagagtgtgtggagcaagctgcagagcattggaatcaggcaacagcttgagagagttcagctacgggaactgtcacaagaggaggtcaagcatcaccaggacacttggctggccatgcctatctgcagattgcgcttcatccccaagctcaatggtctccggcccattgtgaacatgagttatggcatggacaccagagcttttggcaaaaagaagcagacccagtgtttcactcagagtctcaagactttgttcagcgtgctcaactacgagcggaccaaacatcctaaccttatgggtgcttcagtactgggtacgagtgacagctacaggatctggcggaccttcgtgctgcgtgtgcgtgctctggaccagacacccaggatgtactttgttaaggcagatgtgacaggggcctatgatgccatcccccaggacaagctcgtggaaattgtcgccaatataatcaggcgctcagagagcatgtactgtatccgccagtatgcagtggttcagaaagatagccaaggccaagtccacaagtccttcaggagacaggtctccaccctctctgacctccagccatacatgggccagttcaccaagcatctgcaggactcagatgccagtgcactgaggaactctgttgtcatcgagcagagcatctccatgaatgagactggcagtagcctgctccacttcttcctgcgctttgtccgtcacagtgtcgtgaagatcgatggcaggttctatgtgcaatgccagggcatcccccagggctccagcctgtccaccctgctctgcagtctgtgtttcggagacatggagaacaagctgtttgccgaggtgcagcaggacggcttgcttttacgttttgtcgatgactttctgttggtgacacctcacctggcccatgcaaaagcctttctcagcaccctggtccatggcgtgcccgagtatggctgcatgataaacttgcagaagacagtggtgaacttccctgtggagaccggcgccctgggaggtgcagccccgcaccagctgcctgctcactgcctgtttccctggtgtggcttactgctggacactcggactttggaagtattctgtgactactcaggttacggacggacctcaattaagatgagcctcaccttccagggtgtctccagggccgggaagaccatgcggtacaagctcttgtcagtcttgcggttgaagtgtcatggtctgtttctagacttgcaggtgaacagcctgcagacagtctgcatcaatatatacaagatcttcctgcttcaggcctacaggttccatgcatgtgtgattcggcttccctttggccagcatgttaggaagaaccatgcattctttctgggcatcatctccaacctagcatcctgctgctacgccatcctgaaggtcaagaatccaggagtgtcactaagggccaagggtgcccctggctcctttccgcccgaggccacacgttggctctgctaccaagccttcctgctcaagctggctgctcattctgtcacctacaagtgtctcctgggacctcttaggacagcccaaaaacagctgtgccggaagctcccagaggcaacaatgaccctccttaagactgcagctgacccagccctaagcacagattttcagaccattttggactaa

SEQ ID NO: 15 - DNA, 1903 bp, Homo sapiens
cggcgtccccggggcccactcccgagcgcaggcgggcagccaggcgggcggcgcggcgcgggccggcaggaagcgtattctgggcacggggcgccgggcgggccggctgcgccgagcggcagtggtgggataccacccaaggcctcgcgcggcgccgcccgtcgaggggcgggcggcggcgtagccactgggccgtcgaagagcgcaggaggccggtgggccgggccgggccgcgcggcgcagccatgcctggctttacgtgctgcgtgccaggctgctacaacaactcgcaccgggacaaggcgctgcacttctacacgtttccaaaggacgctgagttgcggcgcctctggctcaagaacgtgtcgcgtgccggcgtcagtgggtgcttctccaccttccagcccaccacaggccaccgtctctgcagcgttcacttccagggcggccgcaagacctacacggtacgcgtccccaccatcttcccgctgcgcggcgtcaatgagcgcaaagtagcgcgcagacccgctggggccgcggccgcccgccgcaggcagcagcagcaacagcagcagcagcagcaacagcagcaacagcagcagcagcagcaacagcagcagcagcagcagcagcagcagcagtcctcaccctctgcctccactgcccagactgcccagctgcagccgaacctggtatctgcttccgcggccgtgcttctcacccttcaggccactgtagacagcagtcaggctccgggatccgtacagccggcgcccatcactcccactggagaagacgtgaagcccatcgatctcacagtgcaagtggagtttgcagccgcagagggcgcagccgctgcggccgccgcgtcggagttacaggctgctaccgcagggctggaggctgccgagtgccctatgggcccccagttggtggtggtaggggaagagggcttccctgatactggctccgaccattcgtactccttgtcgtcaggcaccacggaggaggagctcctgcgcaagctgaatgagcagcgggacatcctggctctgatggaagtgaagatgaaagagatgaaaggcagcattcgccacctgcgtctcactgaggccaagctgcgcgaagaactgcgtgagaaggatcggctgcttgccatggctgtcatccgcaagaagcacggaatgtgaactggtgccccggcagcctgctggactcccagaccccatccagccaggggaccgcaggccattgttgaactcctctatactcctgggcactggttgacagtactgaggcttaaggcagctggactctcttgctggtgacctggcatcctcaattgtttcctcctgaagtggaagctggggccttagactctgccctggtgacaccagcaattatgactttgtctacccttcctccccagttattgttgcagattctggttaagcagaggcttcagaaccactgaacttgaaacttaccctctagggatgcaggtgggatgtccagggactataggtttgggaaaaccataccttaaggttggtcagcagtcagacaactctaatgtgtgtagtgataagagattcaagtaacatcagttctcctccttttcatgcttttccttcccaggtgcagcctgtgattctgatggggactggtaaatctgtgcctctgcctcctaggacttattttcccaggaggccatttacaaggggatctggatgacctgctgatggagatccagcttgccagggacttaggtttatcctgttttgtttgctactggttacaaattctattttctgtacaattagtcagactaaagttttcactgtgtttgtttggcaaaacaaattaaacaaaaagtaaggtttttaaaaaaaaaaaaaaaaaa

SEQ ID NO: 16 - DNA, 1832 bp, Mus musculus
gaagcgcaggcggacggccgggtgggtagcagcggcgcgggcggtccgcaaccatattctgggcacggggcctcggcccgggacgactgcgccgggcggcaatagtgagaggcctctgagagcctcgcgcggcgccgcccgtcgaggagctgagacaggggccagacccggccgtcgaagagctcgagaggccggtgggccgggccgcgcgtcgcagccatgcctggctttacgtgctgcgttccgggctgctacaacaattcacaccgggacaaggcgctgcacttctacacgtttcccaaggacgctgagttgcggcgcctctggctcaagaacgtgtcccgtgctggcgtcagtgggtgcttctccaccttccaacccaccaccggccaccgtctctgcagcgtccactttcagggcggccgcaagacctacacggtgcgcgttcccaccattttcccgctgcgtggcgtcaatgagcgcaaagtagctcggagacctgcgggagctgcggcagcccgccgtaggcagcagcagcagcaacaacaacagcagcagcagcaacagcagcagctgcaacagcagcagccgtctccgtcctcctccactgcccagaccacccagttgcagccgaacctggtgtctgcctctgcagctgtgcttcttacgcttcaggccgccgtagacagcaaccaggctccgggatccgtggttcccgtgtccacgactccctcgggagatgatgtgaagcccatcgacctcacagtgcaagtcgagtttgcagctgctgaaggggcagccgccgctgccgccgcctcagagctagaggctgctacggctgggctggaggccgctgagtgcactctgggccctcagctggtggtggtaggggaagagggcttccctgacactggctctgaccactcgtactccttgtcctcgggtaccacggaggaggagctcctgcgcaagctgaacgagcagcgggacatcttggccctgatggaagtgaagatgaaggagatgaagggtagcatccgccatctgcgtctcaccgaggccaagctccgtgaagaacttcgagagaaggatcgtctgcttgccatggctgtcatccgtaagaagcacggcatgtgaatggttccccccagaaacctgcagaattcgtgactccttccagcccaaggaatcctcaggcaaatgttgtactccccagtattcctgttgacagtgccgaggtttaggacagcgggactccagttggtcagttggcaccttctttcgtctcctcgtgaagtaagagttggggacttcagaatctggtgacaccagcagttatgactttgtctctcactccccagtttatgttgcagattctggttatacacaggcttcagaaccactgaacttggaacttaccctggagggggtgcagatggaactcttaagggactgtgggtttgaggaaaccacttcttcaggttggccagcaatcagagacagctttgctgtgtgtagtgataagagatctcagtaacatcgtttctcctttccatgctacccggttcaggtgcagtctgtgattctgatggggactagctgatctgtgcctctgtctcttaggacctctctacccagaaggtcatttatgagagggctctgggtgacttgctgatggagatccagctcaccagggacttaggtttatctcgttatgtttgctactggttacaaattctattttctgtacaattagactgaagttttcactgtttggcaaaacaaattaaacaaaaaagtaaggtttttaaaaaaaaaaaaaaaaggccaca

SEQ ID NO: 17 - DNA, 55 bp, Escherichia coli
tctagagcggccgcccggcgggtcgccaccatgagtgtggatccagcttgtcccc

SEQ ID NO: 18 - DNA, 36 bp, Escherichia coli
catgcaacctgaagacgtgtgaagactagtatcgat

SEQ ID NO: 19 - DNA, 51 bp, Escherichia coli
tctagagcggccgcccggcgggtcgccaccatggctgtcagcgacgcgctg

SEQ ID NO: 20 - DNA, 46 bp, Escherichia coli
ccacctcgccttacacatgaagaggcatttttaaaagcttatcgat

SEQ ID NO: 21 - DNA, 50 bp, Escherichia coli
tctagaggatccccggcgggtcgccaccatggcggccgcgggaattcgat

SEQ ID NO: 22 - DNA, 39 bp, Escherichia coli
ggcacactgcccctctcacacatgtgaaagcttatcgat

SEQ ID NO: 23 - DNA, 51 bp, Escherichia coli
tctagagcggccgcccggcgggtcgccaccatggcgggacacctggcttcg

SEQ ID NO: 24 - DNA, 37 bp, Escherichia coli
gggctctcccatgcattcaaactgaggatccatcgat

SEQ ID NO: 25 - DNA, 50 bp, Escherichia coli
tctagagcggccgcccggcgggtcgccaccatgccgcgcgctccccgctg

SEQ ID NO: 26 - DNA, 41 bp, Escherichia coli
caaaatcccaagcgtggcgctgcccttaaaagcttatcgat

SEQ ID NO: 27 - DNA, 43 bp, Escherichia coli
tctagagcggccgcccggcgggtcgccaccatggtgagcaagg

SEQ ID NO: 28 - DNA, 38 bp, Escherichia coli
ctcggcatggacgagctgtacaataaaagcttatcgat

SEQ ID NO: 29 - DNA, 22 bp, Homo sapiens
gaggatcaccctgggatataca

SEQ ID NO: 30 - DNA, 22 bp, Homo sapiens
agatggtcgtttggctgaatac